(19) United States
(12) Patent Application Publication
(10) Pub. No.: US 2026/0204378 A1
(43) Pub. Date: Jul. 16, 2026

(54) SYSTEMS AND METHODS FOR PATIENT-SPECIFIC TREATMENT RECOMMENDATION OPTIMIZED WITH MULTISTAGE MACHINE LEARNING

(71) Applicant: Mayo Foundation for Medical Education and Research, Rochester, MN (US)

(72) Inventor: Fred M. Cutrer, Rochester, MN (US)

(21) Appl. No.: 19/136,790

(22) PCT Filed: Dec. 12, 2023
(86) PCT No.: PCT/US2023/083698

§ 371 Date: Jun. 6, 2025

Related U.S. Application Data
(60) Provisional application No. 63/387,033, filed on Dec. 12, 2022.

(51) Int. Cl. G16H 20/10; G16B 20/20; G16H 10/20; G16H 10/60; G16H 50/70

(52) U.S. Cl. CPC G16H 20/10; G16B 20/20; G16H 10/20; G16H 10/60; G16H 50/70

(57) ABSTRACT

An optimal drug for treating a medical condition of a patient is determined from a list of candidate drugs based on patient health data acquired from the patient. A multiphase drug recommendation model processes the patient health data to reprioritize a list of candidate drugs, thereby determining an optimized drug for the particular patient. In one phase, the drug recommendation model is updated with predictive scores generated using a multistage machine learning framework, in which the patient health data is used to generate predictive scores that indicate positive and/or negative predictors of therapeutic efficacy and/or tolerability of a candidate drug.

### CROSS-REFERENCE TO RELATED APPLICATIONS

This application claims priority to U.S. Provisional Application No. 63/387,033, filed Dec. 12, 2022, the entire contents of which is incorporated herein by reference.

### BACKGROUND

In the current standard practice for the treatment of a chronic disorder, at the initial visit, patients arrive and give information about their disease, a diagnosis is made, and they are started on a course of treatment. This initial visit is documented in a note, through dictation or typed into the medical record. The initial note is placed into the electronic medical record (“EMR”). There is little consistency in the details of the information that is gathered and almost none of the information is retained in a manner that allows analysis across patients who share the same disease.

Under current standard practice, patients receive treatment for a period of time and then return for assessment of the effectiveness of the treatment occasionally based on changes in imaging or laboratory tests, however, in many instances treatment response is based on the patient's self-report or, at best, on a diary that they are asked to maintain (which may or may not be completed on a daily basis). The response to treatment is documented in the EMR in a text based note. It is not maintained in a form that allows searches for patients with similar patterns of response.

Currently, a note is sent into the EMR. If the patient is a non-responder the treatment is changed, and the foregoing process is repeated until an effective, well-tolerated treatment is identified.

There remains a need to provide a framework for facilitating the digitization of all clinical phenotypic data, to link these data with biorepository data, and to utilize these digitized data for the optimal recommendation of potential treatment options for patients suffering from chronic medical conditions.

### SUMMARY OF THE DISCLOSURE

The present disclosure addresses the aforementioned drawbacks by providing a method for generating an updated prioritized list of candidate drugs for treating a medical condition of a patient. The method includes accessing, with a computer system, a list of candidate drugs for treating a medical condition. The computer system also accesses patient health data acquired from a patient with the medical condition, and a machine learning algorithm that has been trained on training data to generate predictive scores for reprioritizing candidate drugs for treating the medical condition of the patient. The predictive scores reflect or otherwise indicate at least one of a therapeutic efficacy of the candidate drugs, a safety of the candidate drugs, or a tolerability of the candidate drugs. The patient health data are input to the machine learning algorithm using the computer system, generating output data as predictive scores for reprioritizing the list of candidate drugs. The list of candidate drugs and the predictive scores are input to a drug recommendation algorithm implemented by the computer system, generating an output as an updated list of candidate drugs that is optimized for the patient based on their patient health data.

In some configurations, the machine learning algorithm may be trained on training data using supervised learning.

In some configurations, the patient health data may comprise at least one of phenotypic data or genomic data.

In some configurations, the patient health data may comprise phenotypic data that include questionnaire response data indicating patient responses to a questionnaire.

In some configurations, the medical condition may be migraines or chronic headaches and the questionnaire response data may comprise at least one of a headache frequency reported by the patient, a mean headache functional severity score reported by the patient, a mean headache pain intensity score reported by the patient, and a tolerability score reported by the patient, wherein the tolerability score indicates a tolerability of a presently prescribed drug for treating the medical condition.

In some configurations, the predictive scores may comprise at least one of positive predictive scores that increase a priority of an associated candidate drug in the list of candidate drugs and negative predictive scores that decrease a priority of an associated candidate drug in the list of candidate drugs.

In some configurations, the machine learning algorithm may be trained on a training data set comprising clusters of patient health data corresponding to a study group of patients having the medical condition, wherein the clusters of patient health data are correlated with different characteristics.

In some configurations, the different characteristics may comprise therapeutic efficacy of candidate drugs for treating the medical condition or tolerability of candidate drugs by patients in the study group of patients.

In some configurations, the training data set may be generated by inputting the patient health data for each patient in the study group of patients to an unsupervised learning algorithm, generating an output as the clusters of patient health data.

In some configurations, the machine learning algorithm may be a supervised learning algorithm.

In some configurations, the supervised learning algorithm may be a mixed models algorithm.

It is another aspect of the present disclosure to provide a method for generating a drug selection for treating a medical condition of a patient. The method includes accessing, with a computer system, group medical data acquired from a plurality of patients associated with a group. The computer system also accesses a first machine learning algorithm, where the first machine learning algorithm is an unsupervised learning algorithm. The group medical data are input to the first machine learning algorithm using the computer system, generating cluster data as an output. The cluster data include clusters of group medical data associated with a candidate drug characteristic. New patient health data acquired from a new patient are accessed with the computer system. A second machine learning algorithm is also accessed with the computer system, where the second machine learning algorithm is a supervised learning algorithm. The new patient health data and the cluster data are input to the second machine learning algorithm using the computer system, generating predictive score data as an output. A drug recommendation model is accessed with the computer system, where the drug recommendation model is configured to determine a drug treatment recommendation based on patient health data. The drug recommendation model is updated using the predictive score data, and the new patient health data are input to the drug recommendation model, generating an optimized drug selection for the new patient.

It is another aspect of the present disclosure to provide a method for training a machine learning algorithm for reprioritizing a list of candidate drugs for treating a medical condition based on patient data acquired from a patient with the medical condition. The method includes accessing patient health data acquired from a group of patients having the medical condition and inputting the patient health data to a first machine learning algorithm, with a computer system, using unsupervised learning to generate data clusters that are identified as being associated with characteristics correlated with a candidate treatment or drug. A second machine learning algorithm is then trained on a second training data set using supervised learning. The second training data set includes data clusters generated using the first machine learning algorithm, where the second machine learning algorithm is trained on the second training data set to generate classified feature data indicating a presence or absence of a characteristic associated with the data clusters that is indicative of at least one or therapeutic efficacy or tolerability of a candidate drug for treating the medical condition. The second machine learning algorithm is then stored with the computer system.

It is another aspect of the present disclosure to provide a method for training a machine learning algorithm for reprioritizing a list of candidate drugs for treating a medical condition based on patient data acquired from a patient with the medical condition. The method includes accessing patient health data acquired from a group of patients having the medical condition and inputting the patient health data to a first machine learning algorithm, with a computer system, using unsupervised learning to generate data clusters that are identified as being correlated with characteristics associated with a candidate treatment or drug. The data clusters identified with unsupervised learning are then assessed as to their predictive accuracy in a second training data set using supervised learning. The second training data set includes data from a distinct group of patients in whom the outcome of treatment has been captured and is used to determine which data clusters generated using the first machine learning algorithm are classified feature data indicating a presence or absence of a characteristic indicative of at least one of therapeutic efficacy or tolerability of a candidate drug for treating the medical condition. After the machine learning algorithm is trained for optimal predictive accuracy by supervised learning, it is then stored with the computer system.

In some configurations, the group of subjects may include subjects classified as one of responders or non-responders to at least one candidate drug included in the list of candidate drugs for treating the medical condition.

In some configurations, the group of subjects may include subjects classified as one of tolerators or non-tolerators of at least one candidate drug included in the list of candidate drugs for treating the medical condition.

In some configurations, the first machine learning algorithm may be a k-means clustering algorithm.

In some configurations, the second machine learning algorithm may be a mixed models algorithm.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the technology disclosed herein, however, and reference is therefore made to the claims and herein for interpreting the scope of the technology disclosed herein.

### BRIEF DESCRIPTION OF THE DRAWINGS

FIG. 1A illustrates an example drug recommendation model in accordance with some embodiments described in the present disclosure.

FIG. 1B illustrates another example drug recommendation model in accordance with additional embodiments described in the present disclosure, in which a multistage machine learning framework is used to generate predictive score values to reprioritize a list of candidate drugs for treating a medical condition of a patient.

FIG. 2 is a block diagram of an example drug recommendation system.

FIGS. 3A-3F illustrate example user interfaces according to some configurations.

FIG. 4 is a flowchart illustrating the steps of an example method for constructing or otherwise updating a drug recommendation model for determining an optimized candidate drug and/or dosing for treating a medical condition in a patient.

FIGS. 5A-5B are block diagrams of another example drug recommendation system.

FIG. 6 illustrates an example process for facilitating digitization of functional molecular cascades impacted by a drug according to some configurations.

FIG. 7 illustrates an example process for identifying a relevant mechanism of action in empirically used anti-migraine prophylactic medications according to some configurations.

FIG. 8A illustrates a graph of results associated with Atenolol according to some configurations.

FIG. 8B illustrates a graph of results associated with Topiramate according to some configurations.

FIG. 8C is a graph illustrating a genomic distribution (with respect to Transcription Start Site) of variants associated with treatment response to Atenolol.

FIG. 8D is a graph illustrating a genomic distribution (with respect to Transcription Start Site) of variants associated with treatment response to Topiramate.

FIG. 9 is a block diagram of an example system that can implement the drug recommendation system and multistage machine learning-based drug recommendation optimization described in the present disclosure.

FIG. 10 is a block diagram of example components that can implement the system of FIG. 9.

### DETAILED DESCRIPTION

Described here are systems and methods for determining an optimal drug, or other treatment, type recommendation for a patient based on patient health data, which may include electronic medical record (“EMR”) data, electronic health record (“EHR”) data, drug and/or treatment tolerability data, comorbidity data, concurrent medication and/or treatment data, and the like. The drug recommendation system implements one or more artificial intelligence (“AI”) models to determine the optimal drug type. The AI models may in some instances include one or more machine learning models that may be trained and/or retrained using different learning techniques, including unsupervised and supervised learning. It is an advantage that using machine learning or other AI models, the disclosed drug recommendation systems and methods can be updated in a continual fashion to assess treatment successes and/or failures. For instance, machine learning or other AI models can be used to identify data characteristic that correlate with success and/or failure of the treatment recommended by the disclosed systems and methods. These identified data characteristics or elements can be used to continually optimize the predictive recommendations of the disclosed systems and methods, as described below in more detail.

In general, the systems and methods described in the present disclosure determine a drug type and/or dose that is optimized for a particular patient to treat a particular medical condition. As a non-limiting example, the medical condition can be migraines, chronic headaches, or the like. In other examples, the medical condition can be other chronic illnesses or medical conditions.

As one non-limiting example, the disclosed systems and methods provide an analytical framework for digitizing and parsing through patient health data (e.g., results of drug trials to treat the particular medical condition) in order to determine an optimal drug selection to administer to the patient and/or to identify new generations of drugs that could be administered to the patient to optimally treat the particular medical condition.

Advantageously, the disclosed systems and methods analyze the patient's own health data, such that an individually tailored selection for the optimal drug and/or optimal dosing of the drug can be made. As noted above, a drug recommendation system in accordance with some embodiments described in the present disclosure includes a multilayer, or multistep analysis framework. The drug recommendation system has a stored prioritized list of drug or other treatment options. In use, a patient will complete a questionnaire and the data from that questionnaire will be input to the drug recommendation system. The questionnaire response data may be stored as a part of the patient health data for that patient, which may include storing the questionnaire response data as part of the patient's electronic medical record, or in another connected database or data storage device. The questionnaire response data are utilized to pare down or otherwise reduce the drug options in the stored prioritized list of drug options for the particular medical condition.

In general, the drug recommendation systems described in the present disclosure offer patients a lower cost option for routine medication titration follow-up visits, while still retaining the intrinsic value of the visits. The drug recommendation system can, in some embodiments, be implemented using internet-based or other remote visits conducted by mid-level care providers supported by an algorithm-based tool. The tool replicates the multi-factorial processing carried out by sub-specialty physicians in selection of the next prophylactic medication to be tried.

The process generally starts with a face-to-face consultation with the sub-specialty physician. At that time, a comprehensive history review and neurological examination can be carried out. Diagnostic testing, if any, is ordered and appropriate actions are taken for any clinically significant test results. Once this process is completed, if management of the patient's medical condition is required, an initial treatment plan is recommended and the patient is given the treatment for a first duration of time (e.g., 12 to 14 weeks). Under current practice, the patient must return for a face-to-face visit with the physician or a mid-level practitioner at the end of this treatment epoch to assess the patient's response to the therapy. If the patient has not responded to, or has been intolerant of, the medication, the patient is offered a new treatment that works by a different mechanism of action. The patient then must return again for reassessment of their response to the new treatment after another duration of time (e.g., another 12 to 14 weeks). This process, which must be repeated until an effective and well-tolerated treatment is found, is often quite costly to patients and disruptive to their family life.

Using the drug recommendation systems described in the present disclosure, the default for these visits can be scheduled internet-based, or other remote, visits, carried out by mid-level providers supported by an algorithm-based system that considers multiple factors and provides to the mid-level providers the top preferred option for the next recommended treatment.

The drug recommendation process is generally as follows. The patient completes a comprehensive initial pre-visit questionnaire, in which the patient reports all of the previous prophylactic medications that they have tried, including what the maximum dose was that they attained, the duration of their treatment at the maximum dose and the occurrence and identification of any limiting drug side effects. The patient's responses to the questionnaire can be stored as questionnaire response data for subsequent use by the drug recommendation model. If the patient is unable to provide this information, the drug response is considered indeterminate and the currently prescribed drug, a previously prescribed drug, or a similar drug, can be retried. In addition, at the first visit the patient can be asked to provide a blood sample for a pharmacogenetic screening test to aid in identification of potential drug interactions and to help determine the optimal dose of any drugs that are being considered for treatment. The results of the screening test can be stored as part of the patient health data for subsequent use by the drug recommendation model.

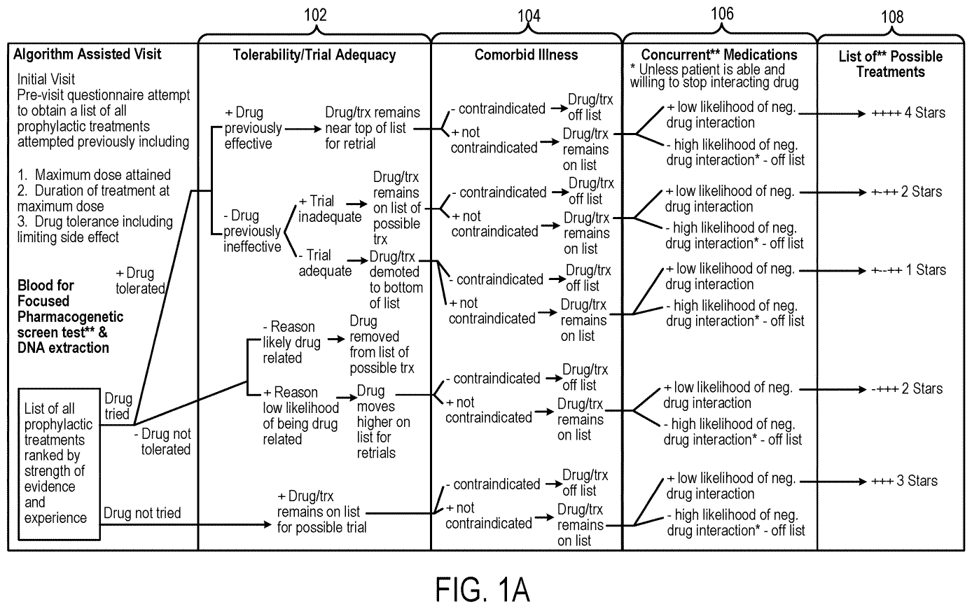

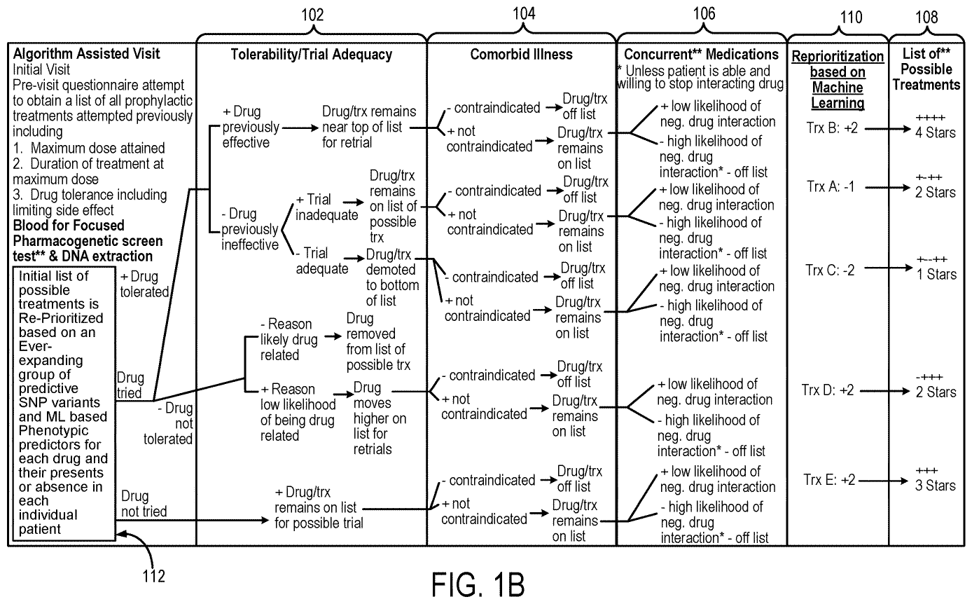

An example drug recommendation program, algorithm, or model that can be implemented by the drug recommendation systems described in the present disclosure is shown in FIG. 1A. The algorithm begins by accessing a list of potential treatments for the patient's medical condition. For example, if the patient's medical condition is chronic migraines, then the drug recommendation algorithm can include a step of accessing a list of potential migraine treatments, which may include all potential migraine treatments, in order of priority based on evidence from the literature and/or clinical experience. Each drug in the retrieved list is filtered by the algorithm through a series of phases, layers, or steps based on factors that can determine whether the medication is suitable for the patient, and if the medication is suitable, whether it is more or less desirable than other potential drugs for the individual patient based on factors specific to the patient.

If the drug has been tried before, it is filtered through a first phase 102. In this first phase 102, the tolerability and/or trial adequacy of the drug is analyzed based on patient health or other data to determine if the previous therapeutic trial was adequate based upon the maximum dose attained and the duration of treatment at the maximum dose. If the previous trial of the drug is deemed adequate, and it was effective for the patient, the drug passes to the second phase 104. If the trial of the drug was deemed adequate, but the drug was ineffective, it is taken off the list for the patient. If the trial of the drug was deemed inadequate, the drug stays on the list of possible treatments and is passed to the second phase 104. If the drug has not been tried at all, it passes directly to the second phase 104. If the drug remains on the list, it moves to the second phase 104, in which comorbid illnesses are analyzed.

In this second phase 104, if the drug is contraindicated in a patient with the comorbid disorder, it is removed from the list for the patient. If the drug is only relatively contraindicated, or otherwise less desirable in a patient with the comorbid disorder, it remains on the list but is given a demerit (e.g., is ranked or scored lower than other potential drug options in the list), lowering its priority position on the list relative to the other competing treatments. Drugs remaining on the list move to the third phase 106, in which concurrent medications are analyzed.

In the third phase 106, all concurrent medications, including over the counter medications and dietary supplements, are assessed as to their likelihood of having a negative drug interaction with each possible drug still on the list. If a candidate drug is determined to have a high likelihood of having a serious interaction with a concurrent medication (e.g., based on a comprehensive, routinely updated drug-drug interaction database), the candidate drug is taken off the list of available treatments for the patient unless the patient is able and willing to discontinue the interacting drug. As a non-limiting example, the drug-drug interaction may pertain to known effects of the candidate drug that may adversely increase the effect of or undermine the effectiveness of the patient's concurrent medication(s).

In addition, information from the pharmacogenetic screen (e.g., data provided as part of the patient health data input to the drug recommendation system) is implemented to determine whether the patient carries variants that can predict whether the patient is likely to have an interaction with a concurrent medication based on altered metabolism of the two medications. When the patient has such a variant, the candidate drug is taken off the list, or its priority is lowered depending on whether the potential metabolism-based interaction is serious or mild. If the potential medication is determined to have a low likelihood of a negative drug interaction and the patient does not carry a gene variant that suggests they will have a negative interaction, the candidate drug remains on the list and moves into the fourth phase 108.

In the fourth phase 108, all remaining possible treatments are listed in order of suitability for the patient. As a non-limiting example, each remaining candidate drug can be ranked with a corresponding score, such as a 1- to 5-star rating. If none of the remaining candidate drugs are ranked above a selected threshold (e.g., a 2-star rating), a non-pharmacological treatment option can be suggested as a viable treatment option. For example, in the case where the patient's medical condition is chronic migraines, a non-pharmacological treatment option may include a supraorbital nerve stimulator, a vagal nerve stimulator, or a transcranial magnetic stimulator.

The practitioner, before prescribing a particular candidate drug from the output list can retrieve (e.g., via a user interface of the drug recommendation system) a list or set of factors or other data that contributed to the ranking or order of the candidate drug in the list. In the priority list, if the information from the pharmacogenetic screening test indicates that the patient carries a variant that would alter their metabolism of the candidate drug, appropriate dosing adjustments can be made based on this feedback provided by the drug recommendation system. Additionally, or alternatively, the practitioner can initiate an eScript for signing and issuing a prescription of the selected candidate drug to the patient.

In some implementations, concurrent with the fourth phase 108, the drug recommendation model can retrieve and identify insurance information stored in the patient's electronic medical record and determine which of the candidate drugs are on the formulary list for the patient's insurance policy. The drug recommendation system can retrieve this information and provide it to the practitioner in order for the treatment to be pre-authorized and to provide that information at the time of drug selection.

As will be described below in more detail, the systems and methods can further include a multistage machine learning framework for analyzing patient health data to aid in generating a recommendation for an optimized drug and/or dose for the patient, which may include updating a drug recommendation program, algorithm, or model based on the output of one or more machine learning algorithms or models. For instance, as shown in FIG. 1B, the drug recommendation system can include an intermediate phase 110 corresponding to the multistage machine learning optimization of drug recommendations. In this intermediate phase 110, the candidate drugs remaining in the list after the third phase 106 can be reprioritized based on the output of the multistage machine learning framework, such as by weighting the candidate drugs using predictive scores generated by the multistage machine learning framework.

Advantageously, machine learning can be used in this way to allow for the efficacy and output of the drug recommendation program, algorithm, or model to be regularly improved. The multistage machine learning framework can be incorporated into the drug recommendation system and applied to patient health data sets for individuals in which treatment outcomes from the drug recommendation system have been captured. As noted above, it is in this intermediate phase 110 that a self-learning aspect of the algorithm optimizer (e.g., a machine learning or other AI model) can be applied to identify factors that are associated with therapeutic success or failure in treatments that have been previously suggested by the drug recommendation systems and methods. For instance, in the intermediate phase 110, such factors can be identified as present or absent in a particular individual and can therefore be used in a reprioritization of the candidate treatments for that individual before drug candidate recommendations are presented to a clinician.

In a first stage, data exploration is performed across data sets for every patient in a study group. As a non-limiting example, an unsupervised machine learning algorithm or model can be used in this first stage. For instance, a k-means clustering or other clustering algorithm can be used to generate data clusters from the study group data sets. The dataset from every individual within a specified study group of interest is subjected to unsupervised machine learning to look for data clustering among the complete phenotypic/genotypic datasets of the study group members.

In a second stage, validation and testing of the data clusters can be performed. For instance, the data clusters can be analyzed using a machine learning algorithm or model to assist in identifying characteristics in the data clusters that are indicative of therapeutic efficacy, drug tolerability, or both. As a non-limiting example, a supervised machine learning algorithm or model can be used in this second stage. For instance, mixed models can be used. New patient health data can be input to such a supervised machine learning algorithm or model in order to generate output data as classified feature data that indicates an absence or presence (e.g., a binary classification) of one or more characteristics in the patient health data that are shared with the study group data and that are indicative of therapeutic efficacy and/or tolerability.

Thus, in this second stage, these data clusters, after validation testing, are then used in a supervised machine learning algorithm, which first uses the data elements implicated as being linked to the shared characteristic to develop a training set. The trained machine learning algorithm is then tested in a new set of individuals to determine the accuracy with which it can predict the presence or absence of the characteristic shared in the study group of interest. The shared characteristic defining the study groups of interest can be, for example, the therapeutic efficacy and tolerability of the recommended treatment. However, other characteristics can also be implemented.

In a third stage, a drug recommendation system can be updated based on the output from the second stage. For instance, those implicated data elements with an accuracy at or above a selected threshold (e.g., 80% or greater) can be employed to alter initial prioritization of drugs in a candidate drug list in the drug recommendation model, as indicated at block 112 in FIG. 1B. As an example, a predictive score for one or more drugs or other treatments can be updated in the drug recommendation system. The predictive score may include a sum of positive predictors, a sum of negative predictors, or a sum of both positive and negative predictors. In some implementations, the predictive score may include a weighted sum of such predictors. The predictive scores can then be used by the drug recommendation system to weight different drug options in the initial priority rank list, thereby improving the optimization of the drug recommendation for a patient based on their own patient health data.

For example, when an individual carries a variant or cluster of phenotypic characteristics that predict response and/or tolerance for a particular treatment, then the priority list in the initial phase will be re-ordered, and the drug may have the appropriately weighted positive value added to the initial prioritization list of the drug by the algorithm. Similarly, when an individual carries a negative predictive variant or phenotypic element(s), then the appropriately weighted negative value may be employed to change the initial prioritization score or ranking for that drug in the initial priority list in the initial phase for the drug recommendation model.

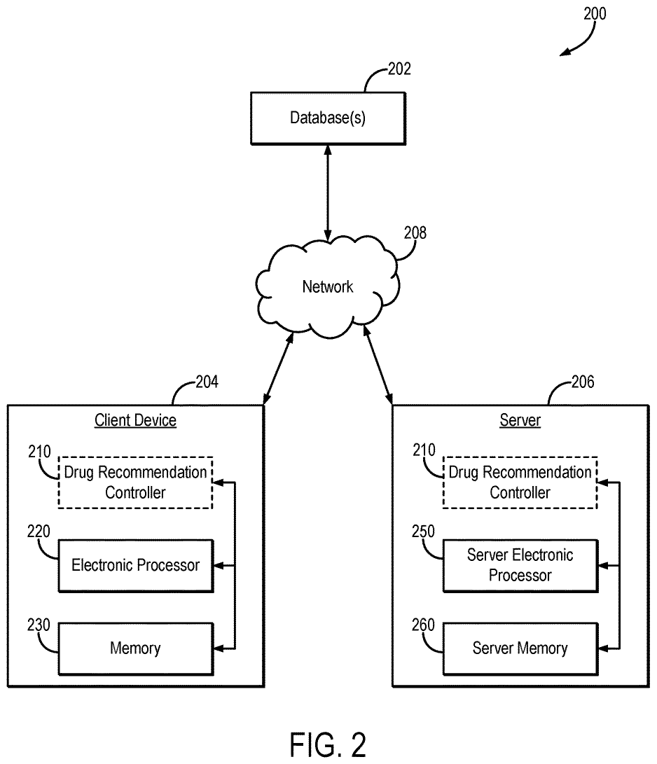

FIG. 2 illustrates an example drug recommendation system 200 that can be implemented in some embodiments described in the present disclosure. The drug recommendation system 200 includes one or more databases 202, a client device 204, a server 206, and a network 208.

The database(s) 202 include one or more databases, data stores, or other data storage or devices that store patient health data. The patient health data may include data stored in, retrieved from, extracted from, or otherwise derived from the patient's electronic medical record (“EMR”) and/or electronic health record (“EHR”). The patient health data can include unstructured text, questionnaire response data, clinical laboratory data, histopathology data, genetic sequencing, medical imaging, and other such clinical data types. Examples of clinical laboratory data and/or histopathology data can include genetic testing and laboratory information, such as performance scores, lab tests, pathology results, prognostic indicators, date of genetic testing, testing method used, and so on.

In some instances, the patient health data can include one or more types of omics data, such as genomics data, proteomics data, transcriptomics data, epigenomics data, metabolomics data, microbiomics data, and other multiomics data types. The patient health data can additionally or alternatively include patient geographic data, demographic data, and the like. In some instances, the patient health data can include information pertaining to diagnoses, responses to treatment regimens, genetic profiles, clinical and phenotypic characteristics, and/or other medical, geographic, demographic, clinical, molecular, or genetic features of the patient.

Features derived from structured, curated, and/or EMR or EHR data may include clinical features such as diagnoses; symptoms: therapies; outcomes; patient demographics, such as patient name, date of birth, gender, and/or ethnicity; diagnosis dates for cancer, illness, disease, or other physical or mental conditions; personal medical history; family medical history; clinical diagnoses, such as date of initial diagnosis, date of metastatic diagnosis, cancer staging, tumor characterization, and tissue of origin; and the like. Additionally, the patient health data may also include features such as treatments and outcomes, such as line of therapy, therapy groups, clinical trials, medications prescribed or taken, surgeries, radiotherapy, imaging, adverse effects, and associated outcomes.

Patient health data can include a set of clinical features associated with information derived from clinical records of a patient, which can include records from family members of the patient. These clinical features and data may be abstracted from unstructured clinical documents, EMR, EHR, or other sources of patient history. Such data may include patient symptoms, diagnosis, treatments, medications, therapies, responses to treatments, laboratory testing results, medical history, geographic locations of each, demographics, or other features of the patient which may be found in the patient's EMR and/or EHR.

In some instances, patient health data can include medical imaging data, which may include images of the patient obtained with one or more different medical imaging modalities, including magnetic resonance imaging (“MRI”), computed tomography (“CT”), x-ray imaging, positron emission tomography (“PET”), ultrasound, and so on. The medical imaging data may also include parameters or features computed or derived from such images. Medical imaging data may also include digital pathology images, such as H&E slides, IHC slides, and the like. The medical imaging data may also include data and/or information from pathology and radiology reports, which may be ordered by a physician during the course of diagnosis and treatment of various illnesses and diseases.

As a non-limiting example, epigenomics data may include data associated with information derived from DNA modifications that are not changes to the DNA sequence and regulate the gene expression. These modifications can be a result of environmental factors based on what the patient may breathe, eat, or drink. These features may include DNA methylation, histone modification, or other factors which deactivate a gene or cause alterations to gene function without altering the sequence of nucleotides in the gene.

Microbiomics data may include, for example, data derived from the viruses and bacteria of a patient. These features may include viral infections which may affect treatment and diagnosis of certain illnesses as well as the bacteria present in the patient's gastrointestinal tract which may affect the efficacy of medicines ingested by the patient.

Proteomics data may include data associated with information derived from the proteins produced in the patient. These features may include protein composition, structure, and activity; when and where proteins are expressed; rates of protein production, degradation, and steady-state abundance; how proteins are modified, for example, post-translational modifications such as phosphorylation; the movement of proteins between subcellular compartments; the involvement of proteins in metabolic pathways; how proteins interact with one another; or modifications to the protein after translation from the RNA such as phosphorylation, ubiquitination, methylation, acetylation, glycosylation, oxidation, or nitrosylation.

One or more of the databases 202 may store genomics data that include genomic information that can be, or have been, correlated with the symptoms and medication effect, tolerance, and/or side effect information that may be received from a patient as responses to a questionnaire and stored as questionnaire response and/or phenotypic data. As a non-limiting example, genomics data can be extracted from blood or saliva samples collected from individuals who have also completed one or more questionnaires such that corresponding questionnaire response data is available for the individuals. A deep phenotypic characterization of these individuals can be assembled. As an example, in one large subset, prospectively determined patterns of treatment response after protocoled titrations in various different drugs from distinct classes of treatments have been assembled. For instance, an analysis of Verapamil, (an L-type calcium channel blocker) using whole exome sequencing (“WES”) can be completed following genotyping in a confirmatory cohort.

In some embodiments, the patient health data can include a collection of data and/or features including all of the data types disclosed above. Alternatively, the patient health data may include a selection of fewer data and/or features.

In the illustrated embodiment, the database(s) 202 communicate with the client device 204. The client device 204 may include, for example, a smartphone, a tablet computer, a cellular phone, a laptop computer, and the like. The client device 204 includes an electronic control assembly having an electronic processor 220, a memory 230, and a transceiver. The transceiver allows the client device 204 to communicate with the database(s) 202, the server 206, or both. The client device 204 may also include a drug recommendation controller 210, which may be implemented as a separate electronic controller and/or processor, or as a part of the processor 220.

The client device 204 communicates with the database(s) 202 to receive at least a portion of the patient health data, to receive configuration information for the drug recommendation system 200, or a combination thereof. In some embodiments, the client device 204 bridges the communication between the database(s) 202 and the server 206. That is, the database(s) 202 transmit patient health or other data to the client device 204, and the client device 204 forwards the patient health or other data from the database(s) 202 to the server 206 over the network 208. In other embodiments, the server 206 can communicate directly with the database(s) 202 and may bridge the communication between the database(s) 202 and the client device 204. For instance, the server 206 receives patient health or other data from the database(s) 202 and the server 206 forwards the patient health or other data from the database(s) 202 to the client device 204.

As will be described in more detail below, the drug recommendation system 200 can implement a process for generating an optimized drug recommendation for a patient using the client device 204, the server 206, or a combination thereof. For instance, the drug recommendation algorithm can be implemented by the client device 204 and/or the server 206. In some instances, a portion of the drug recommendation algorithm can be implemented by the client device 204 and other portions of the drug recommendation algorithm can be implemented by the server 206. As a non-limiting example, the server 206 may implement the multistage machine learning framework for generating predictive scores, which can be communicated to the client device 204 (e.g., via the network 208) in order to update the drug recommendation algorithm being implemented by the client device 204.

The server 206 includes a server electronic control assembly having a server electronic processor 250, a server memory 260, and a transceiver. The transceiver allows the server 206 to communicate with the database(s) 202, the client device 204, or both. The server 206 may also include a drug recommendation controller 210, which may be implemented as a separate electronic controller and/or processor, or as a part of the server electronic processor 250.

The server electronic processor 250 receives patient health data and/or other data from the database(s) 202 (e.g., via the client device 204, via the network 208), stores the received patient health data and/or other data in the server memory 260, and, in some embodiments, uses the received patient health data and/or other data for constructing, training, or adjusting a drug recommendation algorithm or model, and/or a machine learning algorithm or model that may in turn be used to optimize a drug recommendation algorithm or model. The server 206 may maintain a database (e.g., on the server memory 260) for containing patient health data, trained machine learning models and/or algorithms, artificial intelligence models and/or algorithms, and the like.

Although illustrated as a single device, the server 206 may be a distributed device in which the server electronic processor 250 and server memory 260 are distributed among two or more units that are communicatively coupled (e.g., via the network 208).

The network 208 may be a long-range wireless network such as the Internet, a local area network (“LAN”), a wide area network (“WAN”), or a combination thereof. In other embodiments, the network 208 may be a short-range wireless communication network, and in yet other embodiments, the network 208 may be a wired network using, for example, USB cables. In some embodiments, the network 208 may include both wired and wireless devices and connections. Similarly, the server 206 may transmit information to the client device 204 to be forwarded to the database(s) 202, or may retrieve information from the database(s) 202 either directly or through the client device 204. In some embodiments, the server 206 bypasses the client device 204 to access the network 208 and communicate with the database(s) 202 via the network 208. In some embodiments, the database(s) 202 may communicate directly with both the server 206 and the client device 204. In such embodiments, the client device 204 may, for example, generate a graphical user interface to facilitate control and programming of the drug recommendation system 200, while the server 206 may store and analyze larger amounts of patient health or other data for future programming or updating of the drug recommendation system 200.

A drug recommendation controller 210 may be implemented on the client device 204, the server 206, or both. The drug recommendation controller 210 may be a separate processor or controller of the client device 204 and/or the server 206, or may be implemented by an electronic processor or controller of the client device 204 and/or the server 206 (e.g., the electronic processor 220 and/or the server electronic processor 250).

The drug recommendation controller 210 may implement a machine learning program, algorithm, or model. In some implementations, the drug recommendation controller 210 may construct a model (e.g., building one or more algorithms) based on example inputs, which may be done using supervised learning, unsupervised learning, reinforcement learning, ensemble learning, active learning, transfer learning, or other suitable learning techniques for machine learning programs, algorithms, or models. Additionally, or alternatively, the drug recommendation controller 210 may modify a machine learning program, algorithm, or model and/or change output thresholds for a machine learning program, algorithm, or model.

The drug recommendation controller 210 can be programmed and trained to determine an optimized drug option for a patient based on patient health data acquired from that patient (e.g., patient health data retrieved from the database(s) 202 or otherwise input to the drug recommendation controller 210). As a non-limiting example, the drug recommendation controller 210 can construct or store a previously constructed drug recommendation program, algorithm, or model, such as the one illustrated in FIG. 1A and/or FIG. 1B. In some implementations, the drug recommendation controller 210 can implement a combination of unsupervised and supervised learning techniques to optimize or otherwise update a previously constructed drug recommendation program, algorithm, or model.

The drug recommendation controller 210 may be configured to implement various different types of artificial intelligence and/or machine learning algorithms or models. For example, the drug recommendation controller 210 may implement decision tree models, random forest models, association rule learning, artificial neural networks, k-means classifiers, k-nearest neighbors (“KNN”) classifiers, support vector machines, clustering, particle swarm optimization, among others.

The drug recommendation system 200 provides clinical practice optimization. For example, the drug recommendation system 200 decreases clerical burden on clinicians as the drug recommendation system 200 generates the office notes for both initial and follow-up visits and provides a framework for electronically-based visits to replace routine, medication titration face-to-face visits. The drug recommendation system 200 can also alert the clinician of what could be potentially life-threatening adverse medication effects, such as, e.g., drug rashes, chest pain, or any other important symptoms specific to a particular practice. At present, there exists no such notification of the appearance of the adverse reactions.

Data elements in the drug recommendation system 200 can be curated and edited by subspecialist practitioners at the visit, which increases accuracy beyond what may otherwise be an un-reviewed questionnaire.

Questionnaire response data can also be obtained from consistent questions and can be segmented, allowing for the generation of algorithms that can be used to identify factorial predictors of therapeutic outcome (e.g., response vs. non-response; tolerance vs. non-tolerance). Genomic data can be employed in the context of canonical and/or protein network analysis to identify the relevant mechanism of action of common empirically-used medications, thereby leading to the generation of newer, more-specific therapeutic targets.

In one example implementation of the drug recommendation system 200, data are designed to flow through and be compatible with any EMR system. For example, the drug recommendation system 200 can be built to be complaint with and to generate office notes within an EMR system in a templated manner that minimizes the typing, dictation, and editing for documentation. A mobile application presented to the clinician via a client device 204 or server 206 can be designed for the EMR system, but the data after curation in a smart form can flow into the EMR clinical note and into the corresponding fields within the respective database 202. The drug recommendation system 200 may be complaint with the EMR, but storage, management, and analysis can occur outside the EMR system.

In some examples, the disease state requires that the metric of therapeutic response be derived from a mobile device-based diary of symptoms. However, the system is not limited to symptoms. The input used to determine therapeutic response could include any reiterative data derived from electronic or laboratory monitoring of the disease state. This automated data management system provides the technical and workflow framework that can be implemented in all medical specialties dealing with any chronic disease process with multiple therapies.

As described above, in some implementations the drug recommendation system 200 can present a questionnaire to a patient, and can receive responses to that questionnaire as questionnaire response data. As one example, the questionnaire response data can include a questionnaire completed by the patient, questionnaires completed by other patients in a similar group or cohort (e.g., patients having a common medical condition), or the like.

The questionnaire can be presented to a patient via the client device 204 (e.g., via a graphical user interface generated by the client device 204). For example, the questionnaire can be provided via an application (“app”) running on the client device 204, which in some instances may prompt daily questions to the patient to evaluate symptoms and medication side effects. When serious symptoms are indicated in a response to the questionnaire, the app can prompt the patient to schedule an earlier appointment, can prompt a nurse to follow up with the patient, and the like.

In some implementations, the questionnaire can be communicated to the patient via the client device 204 ahead of a visit to the clinic. The responsive data can then be transmitted by the client device 204 to the database(s) 202 and/or server 206 and stored as questionnaire response data. Additionally, or alternatively, the responses to the questionnaire can populate a template visible by the clinician and patient during a clinical visit. The results can also be transcribed into visit notes and added to a database 202 of patient data corresponding to the patient's medical condition.

This process makes visit transcription easier, enables better clinician preparation, and allows the growth of a patient health database for a particular medical condition, which can be linked with other patient health data, including omics data such as genomics data.

In one example form, the app can include a user interface that is automatically populated by an electronic pre-visit questionnaire completed prior to the patient's initial visit. The data in the user interface can be reviewed and confirmed in the context of the initial visit and, when accepted by the clinician, is finalized. Once finalized, data from the user interface can generate a templated office note and be transmitted into a large elemental database, which may be one of the databases 202, the server electronic memory 260 of the server 206, or the like. At subsequent visits, a briefer follow-up questionnaire can be presented to and completed by the patient. As a non-limiting example, the follow-up questionnaire may identify: the interval treatment, its dose, perceived medication side effects, and metrics for treatment response versus non-response (e.g., headache frequency over the past 4 weeks and 90 days, the headache driven functional disability (0-3) and pain severity (0-10)). This process can continue until an effective, well-tolerated treatment is identified using the drug recommendation systems described in the present disclosure. The baseline phenotypic elements are identified primarily in the detailed initial questionnaire and the outcome measures of treatment response and tolerability across subsequent visits.

When the patient's medical condition includes chronic migraines or headaches, the questionnaire may include the two following questions, as an example: “Did you have headache today?” and “Did you take an analgesic medication?” If the answer is “no” to both, then there are no further questions that day and the information is transmitted and retained. If the patient had a headache, then branching logic gives the patient additional questions about headache intensity and severity and whether the patient has had any neurological symptoms suggestive of the migraine aura. The patient may also be asked about analgesic medication use. The questionnaire response data provides a pretreatment baseline. When a prophylactic medication is started at the initial visit, the patient may then be asked if they are taking the preventative medication as prescribed. The patient may receive daily prompts while they are being treated for their headaches, and the information may be retained (e.g., via the database(s) 202). And at interval face-to-face or electronic/telephonic visits, the data can be entered into the database(s) 202 and into a note in the patient's EMR.

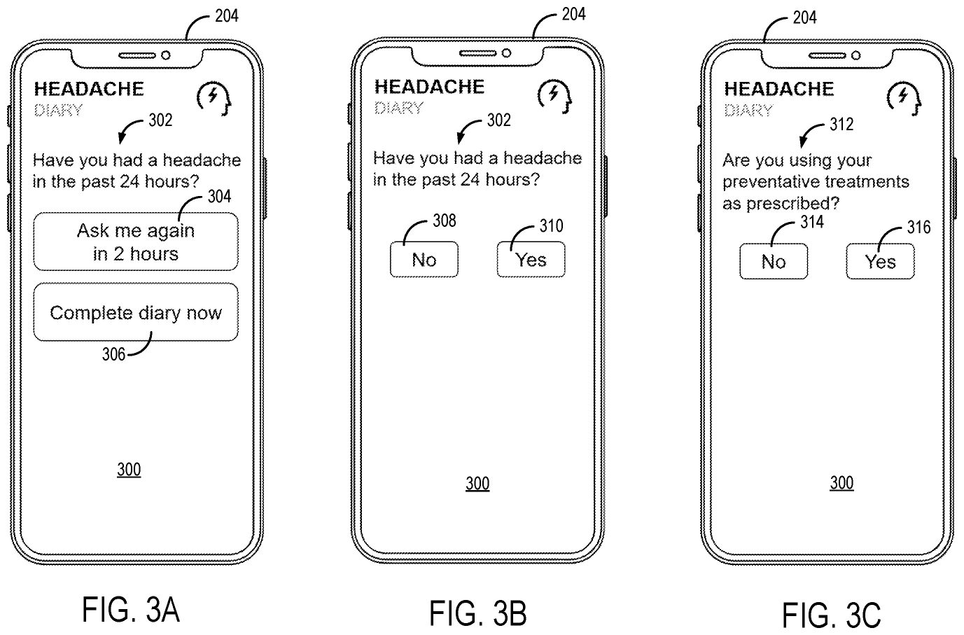

FIGS. 3A-3G illustrate example user interfaces 300 displayed by the client device 204 according to some configurations. In some configurations, the user interfaces 300 may be provided to a user (e.g, via the client device 204) as part of a headache diary (e.g., a questionnaire). As illustrated in FIG. 3A, the user interface 300 may prompt a user for a response to: “Have you had a headache in the past 24 hours?” (represented in FIG. 3A by reference numeral 302). In some instances, the user interfaces 300 may include one or more input elements that the user may interact with in order to provide a response to the prompt. As illustrated in FIG. 3A, the user interface 300 may include a “Ask me again in 2 hours” button 304 and a “Complete diary now” button 306 (as the input elements). When the user interacts with the “Ask me again in 2 hours” button 304, the user may be prompted again in 2 hours. When the user interacts with the “Complete diary now” button 306, the user interface 300 may provide a “No” button 308 and a “Yes” button 310 (as the input elements), as illustrated in FIG. 3B.

As illustrated in FIG. 3C, the user interface 300 may prompt a user for a response to: “Are you using your preventative treatments as prescribed?” (represented in FIG. 3C by reference numeral 312). The user interface 300 may provide a “No” button 314 and a “Yes” button 316 (as the input elements) that a user may interact with to provide a response to the prompt 312, as illustrated in FIG. 3C.

As illustrated in FIG. 3D, after responding to the prompt 312 of FIG. 3C, the user interface 300 may prompt the user for a response to: “Do you need to report headaches that you missed reporting?” (represented in FIG. 3D by reference numeral 318). The user interface 300 may provide a “No” button 320 and a “Yes” button 322 (as the input elements) that a user may interact with to provide a response to the prompt 318, as illustrated in FIG. 3D. When the user interacts with the “No” button 320, the user interface 300 may provide a notification 324 indicating that the user has completed the questionnaire. When the user interacts with the “Yes” button 320, the user interface 300 may prompt the user to provide information for the headaches that were not reported (e.g., via one or more of the user interfaces 300 of FIGS. 3A-3C).

In some instances, when reporting a headache, the application may prompt the user for additional information or details for the headache being reported. For example, as illustrated in FIG. 3E, the user interface 300 may prompt the user for a response to: “How strong was the pain?” (represented in FIG. 3E by reference numeral 326) and/or “How much impact on your ability to function?” (represented in FIG. 3E by reference numeral 328). The user interface 300 may include input elements such that the user may provide a response. For example, the user interface 300 may include one or more checkboxes 330 (as input elements) that a user may interact with to indicate a level of pain associated with the headache being reported, where the level of pain may be rated on a scale from one to ten via checking a corresponding number of the checkboxes 330. As another example, the user interface 300 may include one or more buttons, including a “mild” button 332, a “moderate” button 334, and/or a “severe” button 336, that a user may interact with to indicate an impact that the headache being reported had on the user's ability to function.

Alternatively, or in addition, the user interface 300 may prompt the user for information regarding neurological symptoms associated with the headache being reported. For example, as illustrated in FIG. 3F, the user interface 300 may prompt the user for a response to: “Neurological symptoms with attack?” (represented in FIG. 3F by reference numeral 338). The user interface 300 may include input elements such that the user may provide a response to the prompt 338 via interaction with one or more of the input elements. For example, as illustrated in FIG. 3F, the input elements may include a “None” button 340 (e.g., indicating no neurological symptoms were experienced during the headache being reported), a “Visual loss or distortion” button 342 (e.g., indicating that neurological symptoms related to visual loss or distortion were experienced during the headache being reported), a “Unilateral numbness/tingling” button 344 (e.g., indicating that neurological symptoms related to unilateral numbness and/or tingling were experienced during the headache being reported), a “Unilateral weakness” button 346 (e.g., indicating that neurological symptoms related to unilateral weakness were experienced during the headache being reported), a “Word finding problems” button 348 (e.g., indicating that neurological symptoms related to word finding were experienced during the headache being reported), and/or a “Dizziness or vertigo” button 350 (e.g., indicating that neurological symptoms related to dizziness and/or vertigo were experienced during the headache being reported).

Alternatively, or in addition, the user interface 300 may prompt the user for information regarding treatment of the headache being reported. For example, as illustrated in FIG. 3G, the user interface 300 may prompt the user for a response to: “What treatment did you take to alleviate the pain?” (represented in FIG. 3G by reference numeral 352). The user interface 300 may include input elements such that the user may provide a response to the prompt 352 via interaction with one or more of the input elements. For example, as illustrated in FIG. 3G, the input elements may include a “None” button 354 (e.g., indicating that no treatment was used to treat the headache being reported), a “NSAID” button 356 (e.g., indicating that a non-steroidal anti-inflammatory drug (NSAID) was used to treat the headache being reported), a “Butalbital” button 358 (e.g., indicating that Butalbital was used to treat the headache being reported), a “Tylenol Excedrin” button 360 (e.g., indicating that Tylenol (e.g., acetaminophen) and/or Excedrin (e.g., aspirin/acetaminophen/caffeine) was used to treat the headache being reported), a “CGRP antag” button 362 (e.g., indicating that a calcitonin gene-related peptide (CGRP) antagonist was used to treat the headache being reported), and/or a “Triptan” button 364 (e.g., indicating that Triptan was used to treat the headache being reported).

In some implementations, the client device 204 and/or server 206 can also include an application, program, or algorithm for automating the integration between a phenotypic database (e.g., a database 202 containing questionnaire response data) and genomics data, other omics data, histology data, or other clinical laboratory data that may be stored as a biospecimens accessioning and processing (“BAP”) facility. The functions and characteristics of the data monitoring system are as follows.

The application can monitor the phenotypic database for all initial visit entries and register all new patients. All patients in the database can then be labeled as: “sampled,” “not sampled,” or “needs to be re-sampled.” The application will confirm consent for participation of all willing and/or sampled subjects. The phenotypic database can be monitored by the application for any new treatment response data points that have become available in the interval. Based upon the list of desired phenotypic subpopulations, the application can determine whether or not the data points are usable in any of the pre-specified protocols. When individuals with desired phenotypes are identified, the BAP facility's computerized freezers can be queried. For example, the server 206 can send a query to the BAP facility's computerized freezers, where the query includes: study/protocol ID number, first and last name of the patient, and the like.

Based on the automated queries, a list of all subjects with the phenotypic/treatment response traits specified in each of the pre-specified protocols is maintained and contains all BAP RLIM #'s, volumes, and concentrations of available DNA/RNA or specimens linked to subjects. When the list reaches the number of samples estimated to yield the desired level of statistical significance, the designated subject matter expert is notified, curates the list, and either discards any false positives or requests resampling of any samples deemed insufficient. If the number of samples after curation still meets or exceeds the number likely to have statistical significance, the subject matter expert can have samples pulled from the BAP using data from the BAP query and sends the samples for whole exome sequencing or whole genome sequencing. These clinical data can be used in conjunction with genomic data to assess for biomarkers predictive of the outcome of treatment.

In addition, the algorithm-based queries can assess the phenotypic database for any data element or group of data elements which, within aggregate, correlate with response to therapy.

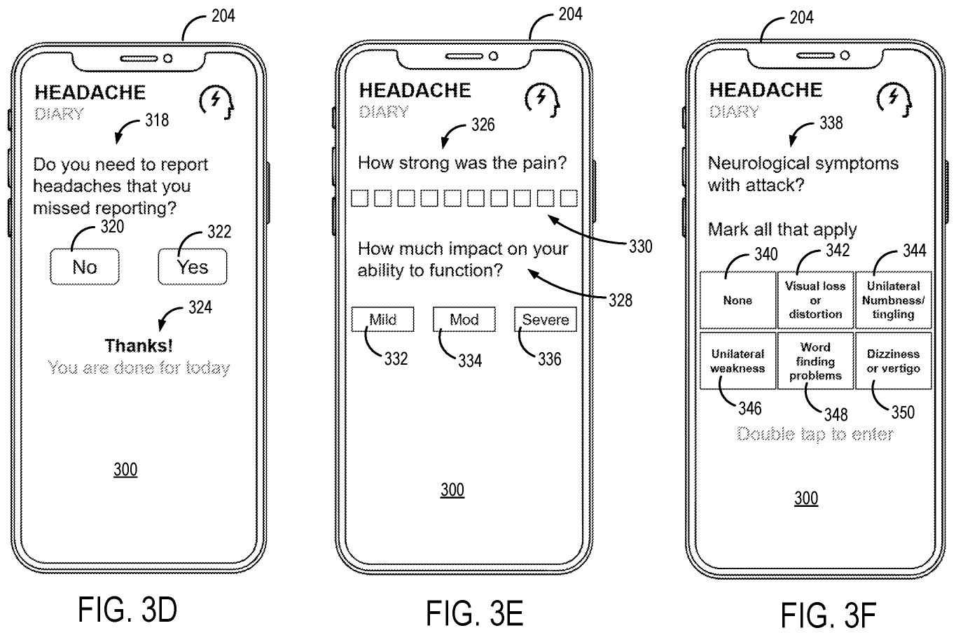

FIG. 4 is a flowchart setting for the steps of an example method 400 for constructing or otherwise updating a drug recommendation model for determining an optimized candidate drug and/or dosing for treating a medical condition in a patient. The method 400 includes accessing patient health data with a computer system, as indicated at step 402. Accessing the patient health data can include retrieving previously acquired medical data from a memory or other data storage device or medium. Additionally, or alternatively, accessing the patient health data can include acquiring patient health data from a patient and recording the patient health data with the computer system, or otherwise transferring the patient health data to the computer system.

As described above, the patient health data can include questionnaire response data and/or other phenotypic data. In some embodiments, the phenotypic data may be linked to other patient health data types. As a non-limiting example, the phenotypic data may be linked with genomic data, such as DNA data. The patient health data can additionally or alternatively include data stored in, retrieved from, extracted from, or otherwise derived from the patient's EMR and/or EHR. The patient health data can include unstructured text, questionnaire response data, clinical laboratory data, histopathology data, genetic sequencing, medical imaging, and other such clinical data types. Examples of clinical laboratory data and/or histopathology data can include genetic testing and laboratory information, such as performance scores, lab tests, pathology results, prognostic indicators, date of genetic testing, testing method used, and so on.

In some instances, the patient health data can include one or more types of omics data, such as genomics data, proteomics data, transcriptomics data, epigenomics data, metabolomics data, microbiomics data, and other multiomics data types. The patient health data can additionally or alternatively include patient geographic data, demographic data, and the like. In some instances, the patient health data can include information pertaining to diagnoses, responses to treatment regimens, genetic profiles, clinical and phenotypic characteristics, and/or other medical, geographic, demographic, clinical, molecular, or genetic features of the patient.

The patient health data are then analyzed with the computer system to extract or otherwise generate medical condition parameters from the medical data, as indicated at step 404. In general, the medical condition parameters can include parameters associated with the clinical presentation of the patient (e.g., parameters associated with one or more reported symptoms) and the tolerability and/or efficacy of the currently prescribed drug treatment for the patient. For instance, the medical condition parameters can be stored as questionnaire response data in the patient health data, or may be derived, computed, or otherwise determined from the patient health data using the computer system.

As a non-limiting example, when the medical condition for which the patient is seeking treatment includes migraines and other chronic headaches, the medical condition parameters may include headache frequency, mean headache functional severity score, mean headache pain intensity score, and tolerability of the currently prescribed drug treatment. Headache frequency can include a qualitative or quantitative measurement of how frequently the patient suffered headaches symptoms over an interval of time. As an example, headache frequency can be computed as the number of days with a headache captured over the trailing 28 days and compared to baseline. Mean headache functional severity score can include values in a range of [1,3]. Mean headache pain intensity score can include values in a range of [1,10]. Tolerability can be a binary value of whether the patient is tolerating the currently prescribed drug treatment, or can be a quantitative value selected from a range of discrete or continuous values that assess tolerability on a scale. Each of these aspects can be considered individually. Additionally, or alternatively, the headache frequency, mean headache functional severity score, and/or mean headache pain intensity score, being continuous variables, can be weighted and combined into a single efficacy value. In some embodiments, the efficacy value can be combined with the tolerability value to generate an outcome factor to assess the success or non-success of a candidate drug (e.g., a currently prescribed candidate drug).

The patient is then added to a patient group based on the medical condition parameters and patient health data, as indicated at step 406. For example, the medical condition data can be used to classify the patient as a responder or non-responder to a currently prescribed candidate drug, and/or as a tolerator or non-tolerator of a currently prescribed candidate drug. The formed patient group(s) encompass a plurality of patients and their corresponding patient health data who have shared patterns of medication specific treatment response (e.g., based on efficacy, tolerability, or outcome factor, which combines both efficacy and tolerability).

The phenotypic data and/or other patient health data for each patient in the patient group can then be mixed and converted to numerical data, as indicated at step 408. For example, binary data values can be converted to 0=absent and 1=present. Categorical data for which there are a limited number of answers can be assigned values based on the available answers. For example, 0=never; 1=formerly present, now absent; 2=present. Continuous data drawn from the patient health data can be converted to a value that is the number's deviation for a normative mean value. For example, BMI=30 can be converted to +8 if BMI of 22 is defined as the mean BMI. If BMI=20, then the value would be −2. Genomic data in the patient health data can be categorical for all SNPs and converted to numerical values. For instance, 0=reference allele, 1=first variant, 2=second variant, etc.

A multistage machine learning framework can then be constructed and implemented to update a drug recommendation model, as indicated at step 410. The multistage machine learning framework can be used to identify the factors (e.g., genomic, phenotypic) that lead to successful treatment of patients in a similar study group. The output of the machine learning algorithm can be used to iteratively optimize the drug recommendation system 200.

The multistage machine learning framework can generally include three stages: a data exploration stage 412 using unsupervised learning, a characteristic determining stage 414 using supervised learning, and a drug recommendation model updating stage 416.

For example, as described above, in the data exploration stage 412 the patient health data for patients in a study group can be assembled as a first training data set for training a first machine learning algorithm, which may be an unsupervised learning algorithm such as a k-means clustering algorithm. Inputting the patient health data from the study group to the first machine learning algorithm generates an output as data clusters, or cluster data, indicative of data that are correlated with therapeutic efficacy, tolerability, or both of a particular candidate drug for treating the medical condition of interest.

In the characteristic determining stage 414, a second machine learning algorithm is trained on a second training data set using supervised learning. The second training data set can include the data clusters generated using the first machine learning algorithm in the data exploration stage 412. This second machine learning algorithm, which may be a mixed models algorithm, is thus trained to generate classified feature data indicating a presence or absence of a characteristic in the data clusters that is indicative of at least one or therapeutic efficacy or tolerability of a candidate drug for treating the medical condition. The patient health data for the patient can be input to the second machine learning algorithm to generate predictive scores or values indicating positive and/or negative prioritization scores of the candidate drug(s).

In the drug recommendation model updating stage 416, the predictive scores output by the second machine learning algorithm are used to update a drug recommendation model. For instance, as described above, positive predictive scores can be used to increase the prioritization of candidate drugs in a list of candidate drugs prioritized by a drug recommendation model, and negative predictive scores can be used to decrease the prioritization of candidate drugs in the list of candidate drugs.

After the drug recommendation system has been updated using the predictive scores generated by the multistage machine learning framework, the patient health data are input to the updated drug recommendation model, generating an output as an updated and/or reprioritized list of candidate drugs for treating the medical condition in the patient, as indicated at step 418.

As an additional advantage, the success or failure of the recommendations for each drug by the drug recommendation system 200 can be assessed and retained digitally by the drug recommendation system 200. Then, factors linked to the therapeutic success or failure of each specific recommended treatment can be investigated using a similar two-step process used to provide the drug recommendation. For example, a discovery phase can be used to identify data clusters related to the success or failure of the recommended drug followed using unsupervised learning. Supervised learning can then be used in a separate group of patients, for whom treatment outcomes of the recommended drug have been recorded. This process can be used to generate factors to increase or decrease the priority score when the drug is about to be recommended in new patients. The output of the optimizing use of this two-step machine learning process can be applied to the final score (e.g., in the intermediate phase 110 the multistage machine learning optimization of drug corresponding to recommendations). This allows the drug recommendation system 200 to continually optimize the success of its predictions with a self-learning, fine tuning iterative process.

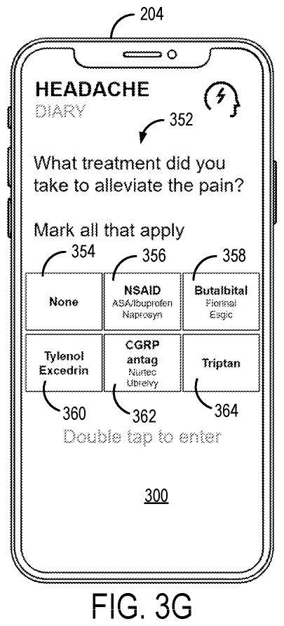

FIG. 5A illustrates another example drug recommendation system 510. The system 510 includes a client 512 that communicates with drug recommendation controller 514 to retrieve a prioritized list of drugs for treating a particular medical condition, retrieve patient health data acquired from a patient with the particular medical condition, and process the patient health data to generate a reprioritized and/or optimized list of candidate drugs. The drug recommendation controller 514 is in communication with several databases, including one or more medical data databases 516, one or more phenotypic data database(s) 518, one or more genomic data database(s) 520, and the like.

In general, the medical data database(s) 516 store medical data, such as medical image data, clinical data, patient record data, histopathology data, or other such medical data. The phenotypic data database(s) 518 store phenotypic registry data, which may include questionnaire response data, as described above in detail. As a non-limiting example, the phenotypic data database(s) 518 can include disease-specific data, whereas the medical data database(s) 516 can include the complete health record for one or more patients. In some embodiments, the phenotypic data database(s) 518 may be a part of the medical data database(s) 516. The genomic data database(s) 520 store genomic data, which may include genomic data that are linked to phenotypic and/or medical data.

As shown in FIG. 5B, communication between the client 512, the drug recommendation controller 514, and the databases (e.g., medical data database(s) 516, phenotypic data database(s) 518, genomic data database(s) 520) can be implemented via a server 524 that is configured to operate as a service layer or middleware. The drug recommendation controller 514 may implement, for example, HL7 standards, DICOM standards, or other suitable standards for various types of medical data.

The client 512 can include a hardware processor, a memory, one or more inputs, and a display. In some examples, the client 512 can include a desktop computer, a laptop computer, a tablet device, a mobile device, or the like. The databases can be any suitable database for storing information such as medical data, including medical images, and associated metadata (e.g., medical data database(s) 516); phenotypic data and associated metadata (e.g., phenotypic data database(s) 518); and/or genomic data and associated metadata (e.g., genomic data database(s) 520). In some examples, the database(s) can implement a SQL database. In some instances, such as when the medical data include medical images, one or more of the databases may be implemented as an archive, such as a Picture Archiving and Communications System (“PACS”), a vendor neutral archive (“VNA”), a long term archive (“LTA”), or other suitable archive for storing medical images and associated metadata on a short-term or long-term basis.

The drug recommendation controller 514 can generally include a hardware processor and a memory. In some implementations, the drug recommendation controller 514 can receive orders from the client 512 via an HL7 standard or other suitable standard, to retrieve patient health data from the database(s) 516, 518, 520 for processing to generate an optimized recommendation for a candidate drug to treat the medical condition of the patient. In these implementations, the drug recommendation controller 514 can retrieve data from the database(s) 516, 518, 520 according to parameters (e.g., patient name or ID, data type, medical condition type) submitted or otherwise queried by the user.

In similar implementations, the drug recommendation controller 514 can receive orders from the client 512 to retrieve data from the database(s) 516, 518, 520 for validation (i.e., a validation work request). In these implementations, the drug recommendation system 514 can retrieve data from the database(s) 516, 518, 520 according to parameters (e.g., patient name or ID, data type, medical condition type) submitted or otherwise queried by the user. The data can then be processed to validate whether the retrieved data are effective for monitoring the therapeutic efficacy and/or tolerability of drugs in the list of candidate drugs.

In some implementations, the drug recommendation controller 514 can locally store a candidate drug list 526 identifying various candidate drugs for treating a particular medical condition and their corresponding doses. A user can query the candidate drug list 526 and select one or more candidate drugs and any corresponding data (e.g., dosing, potential drug interactions, factors used to rank or prioritize the candidate drug). For instance, if the user is a clinician, the user can select candidate drugs from the candidate drug list 526 that have been prioritized for the patient based on an analysis of their patient health data.

The client 512 generally provides a user interface through which a user can communicate requests to the drug recommendation controller 514. For instance, a user can request a reprioritization of the candidate drug list 526 at the client 512 and this work order can be processed by the drug recommendation controller 514 to query the respective database(s), retrieve the relevant data, and process those data to generate an updated candidate drug list 526.

Users can launch an application at the client 512 (e.g., the client application) to both place a new work order and view any outstanding work order requests. Additional views provided on the user interface of the client 512 can include a historical search for viewing and an ability to edit or cancel past work order entries.

Described now are example multiomics approaches to building predictive models, which may be integrated with the drug recommendation systems described in the present disclosure. As noted herein, the technology disclosed in the present disclosure may provide automation of retention of quantitative treatment response data (e.g., therapeutic phenotype) linked to biosamples (e.g., DNA, plasma, and white blood cells) into day-to-day clinical practice. In some configurations, the retained therapeutic phenotype may be the basis of a multiomics approach to building predictive models for drug selection in migraine. Multiomics encompasses genomics, epigenomics, proteomics, metabolomics, microbiomics, and transcriptomics, among other omics. The technology disclosed herein may use the prophylactic drugs as probes to identify biomarkers that are predictive of a positive or negative response to treatment, and, ultimately, improve understanding to pathophysiology of the disease. Accordingly, the technology disclosed herein may provide a process that is a path to development of new drugs with fewer non-beneficial side effects, which can then be given to patients who carry biomarker predictive positive response to the drug.

Many of the drugs which are beneficial in migraine prophylaxis have many biological effects. Adverse effects may be caused by actions of the drug that are irrelevant to migraine treatment. Identification of the effects that drive suppression of migraine may allow the development of optimized forms of the drug without the side effects (e.g., or at least fewer side effects).

In some configurations, the technology disclosed herein may investigate and digitally catalog all the known effects of a specific drug. From this, the molecular pathways impacted by the specific drug can be inferred. Alternatively, or in addition, in some configurations, the technology disclosed herein may investigate and digitally catalog the genetic variation that is a statistically associated response to the specific drug. From this, the molecular pathways impacted by the specific drug can be inferred. Alternatively, or in addition, in some configurations, the technology disclosed herein may investigate plasma metabolomic pre- and post-treatment profiles. The technology disclosed herein may identify metabolite changes correlated with treatment response and with the presence gene alleles most significantly associated with treatment response. In some configurations, the technology disclosed herein may filter for overlap of pathways implicated by genomic variation and metabolomic change in patients responding to the drug and the possible molecular effects of the drug.

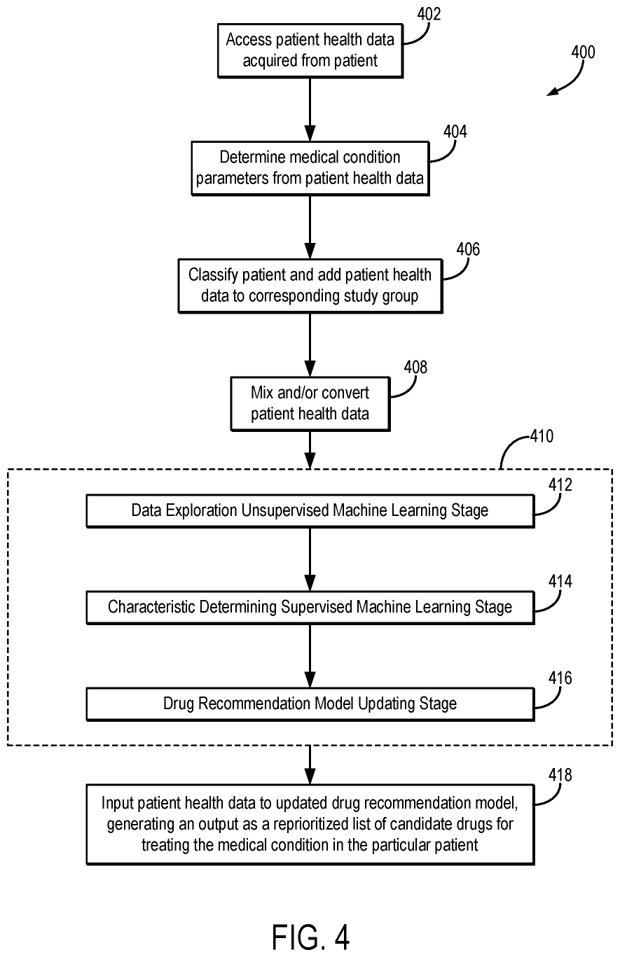

For example, FIG. 6 illustrates an example process 600 to facilitate digitization of functional molecular cascades impacted by a drug according to some configurations. In the example of FIG. 6, the process 600 is described with respect to an example drug referred to herein as “Drug A” (represented in FIG. 6 by reference numeral 605). As illustrated in FIG. 6, at step 610, an AI-driven search may be conducted to identify all known receptor/non-receptor effects of Drug A. In some instances, the AI-driven search may be conducted with respect to one or more pharmacology, medicinal, chemistry, and/or neurochemistry literature repositories and/or other databases. The AI-driven search may be conducted as a keyword search, where the keywords may include, e.g., “mechanism of action,” “target receptor,” “high affinity,” etc.

At step 615, the process 600 may determine other effects of Drug A through interaction with other chemical or physical properties of the body. For instance, a list of other effects of Drug A may be determined based on Drug A's interaction with, e.g., enzymes, ion channels, transporters, etc. At step 620, the process 600 may determine one or more functional molecular cascades impacted by non-receptor effects of Drug A, including, e.g., upstream and downstream first and second degrees. At step 625, the process 600 may determine a list of all target receptors and affinity of Drug A for the receptor. The process 600 may then determine functional molecular cascades for biosynthesis, regulation, and/or degradation of target receptors (at step 630) and determine functional molecular cascades impacted by activation and/or inhibition of target receptors, including, e.g., upstream and downstream first and second degrees (at block 635). At step 640, the process 600 may then determine (or otherwise provide) a digital representation of molecular cascades with Drug A impact (represented in FIG. 6 by reference numeral 650).

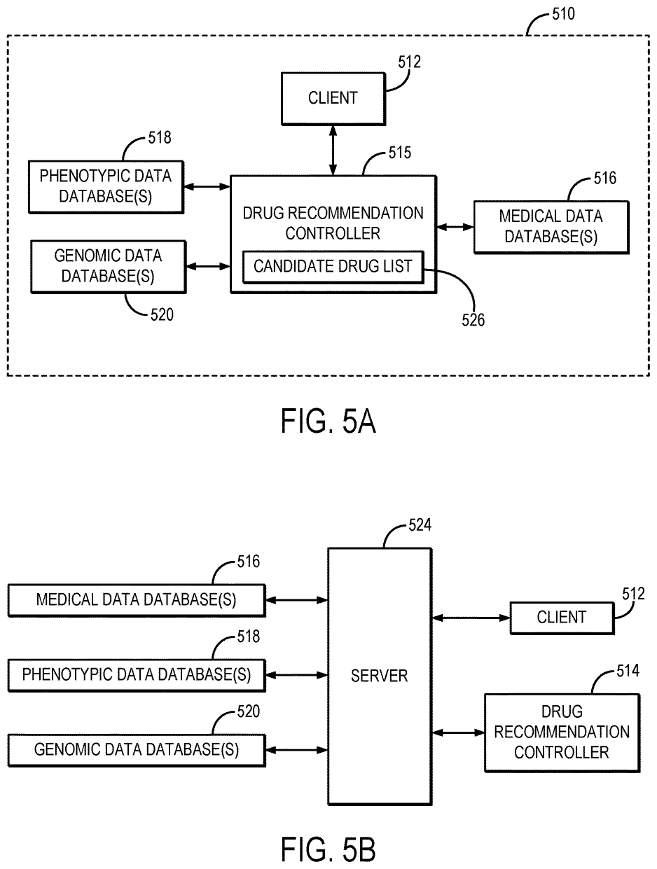

FIG. 7 illustrates an example process 700 for identifying a relevant mechanism of action in empirically used anti-migraine prophylactic medications according to some configurations. In the example of FIG. 7, the process 700 is described with respect to an example drug referred to herein as “Drug A” (represented in FIG. 7 by reference numeral 705).

For example, the process 700 of FIG. 7 may provide a summary schematic for a strategy to identify relevant mechanisms of action in current empirically used anti-migraine prophylactic medications. Such an approach (or process) may include identification of the mechanism from three lines of investigation: (1) an assessment of all possible receptor and non-receptor actions of a drug; (2) an identification of single nucleotide polymorphisms (SNPs) associated with treatment response to the drug and imputation of the functional molecular cascades impacted by the variants; and/or (3) an identification of metabolites whose change is correlated with the presence of SNPs most significantly associated with treatment response, reflecting alteration in the products of the molecular cascades impacted by genetic variation that is correlated with therapeutic success for failure and the potential functional outcome of the alteration. Identification of metabolic patterns associated with treatment response may point to the relevant mechanism of action and lead to easily accessible plasma biomarkers predictive of therapeutic response.

At step 710, the process 700 may perform an assessment of all known receptor/non-receptor effects including first and second degree downstream system affects by Drug A. In some configurations, this assessment may be performed based on an AI-driven search of pharmacology, medicinal, chemistry, and/or neurochemistry databases. At step 715, the process 700 may include an AI-driven search for known functional molecular cascades (canonical and virtual) impacted by Drug A binding or non-receptor effects both direct and downstream. In some configurations, performance of step 715 may result in a digital representation of the known functional molecular cascades (represented in FIG. 7 by reference numeral 718).

At step 720, the process 700 may include storing Patients treated with Drug A DNA and/or extracted and metabolomic or other omic screen pre-treatment. An assessed outcome quantity and/or digitally retrained data (represented in FIG. 7 by reference numeral 722) may be provided for whole genome sequencing (WGS) (represented in FIG. 7 by reference numeral 725).

At step 730, the process 700 may include determining SNPs reaching p<5×10⁻⁸ association with outcome positive (+) or negative (−). At step 735, the process may determine genes impacted by or whose sequence was altered by SNPs associated with Drug A response. The process 700 may include determining pathways containing altered protein gene products or otherwise impacted by variations in associated SNPs (at step 740) and determine first degree downstream pathways impacted by variation (at step 745). In some configurations, performance of step 740 may result in a digital representation of the pathways containing altered protein gene products or otherwise impacted by variations in associated SNPs (represented in FIG. 7 by reference numeral 748). In some configurations, performance of step 745 may result in a digital representation of the first degree downstream pathways impacted by variation (represented in FIG. 7 by reference numeral 750).

At step 755, the process 700 may include performing a metabolomic or other omic screen post-treatment. At step 760, the process 700 may include identifying which serum metabolite or other omic changes correlate with treatment response and with presence of SNP alleles most highly associated with treatment response or non-response. At step 770, the process 700 may determine an overlap of pathways whose metabolite output and function are altered by SNPs associated with Drug A Treatment Outcome in migraine and Functional Pathways impacted or regulated by Drug A. In some configurations, performance of step 770 may result in a digital representation of the overlap of pathway(s) (represented in FIG. 7 by reference numeral 770).

Accordingly, the technology disclosed herein may enable or provide a path to personalized biologically based treatment in migraine. For instance, the technology disclosed herein may enable retention of quantitative therapeutic response data and bio-sampling into routine clinical practice to attain large numbers, use of genomic and/or phenomic approaches focused on therapeutic phenotype to develop predictive models, employ AI-based search of effects of current empiric drugs to infer all known molecular pathways impacted by drug, identify pathways impacted by most significant genomic or other omic variation in patients whose migraine responds to treatment with the drug, filter for overlap of molecular pathways impacted by the drug and varied in treatment responsive patients, etc.

Described now is an example of identification of genomic variants associated with therapeutic response to Atenolol and/or Topiramate in migraine prophylaxis.

The prophylactic treatment of migraine, a disorder that afflicts over 40 million people in the United States, is given to patients with a limited understanding of the pertinent therapeutic drug mechanism(s). Currently there exists no predictive, biologically-based rationale for selecting medications from a pool of at least six pharmacologically distinct therapeutic classes.

In an example study, WGS was performed using the blood DNA samples collected from 370 patients undergoing a three-month monotherapy treatment regimen for migraine prophylaxis, divided between atenolol (n=179) and topiramate (n=191) recipients. The treatment efficacy was assessed using the percentage of headache days reduced (Pre-treatment-Post-treatment/Pre-treatment). Statistical analyses were performed using PLINK and a general linear model. Pathway analyses were then carried out using SNPs associated with either atenolol or topiramate monotherapy treatment.

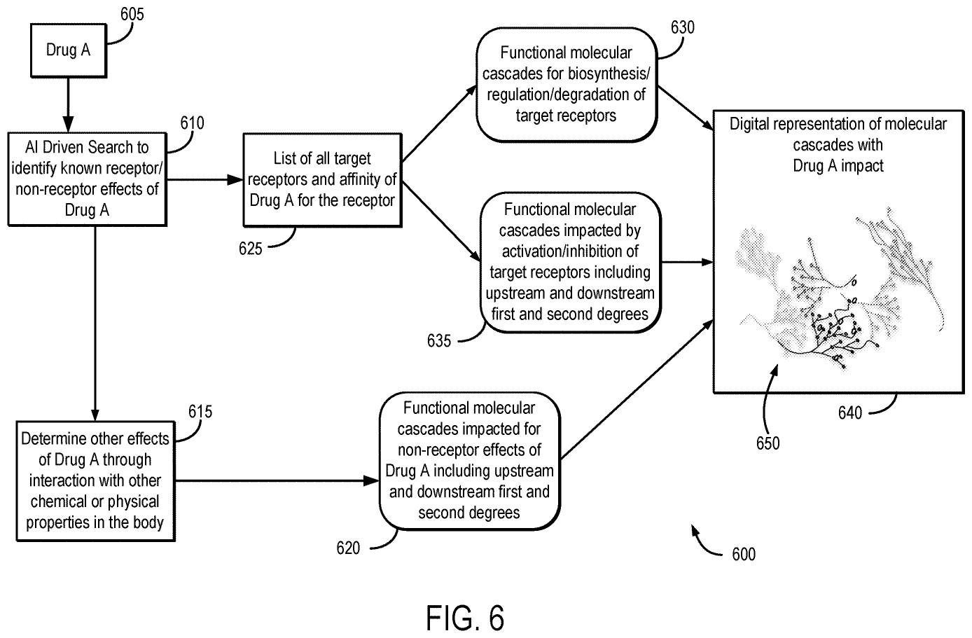

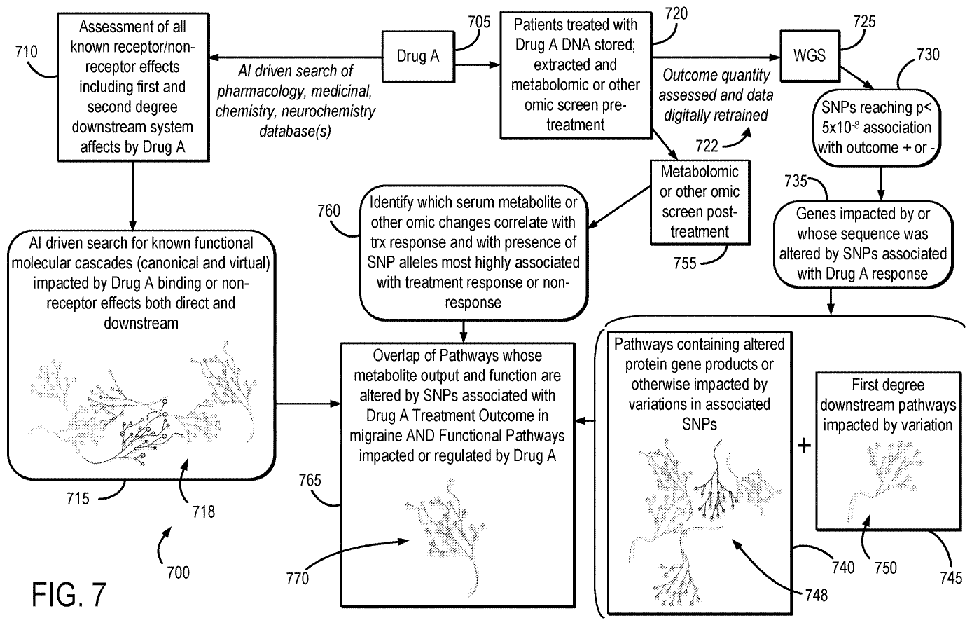

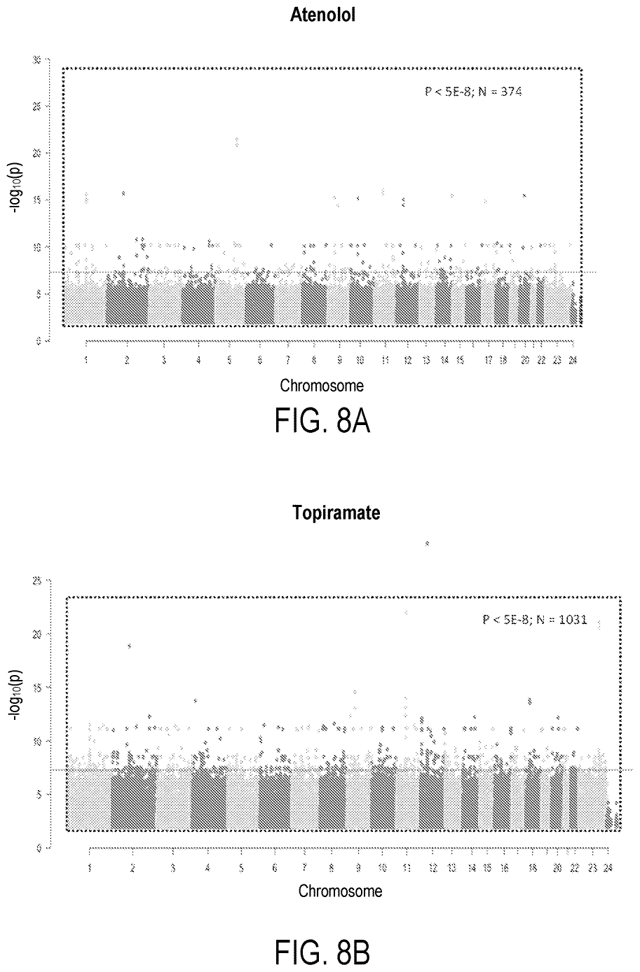

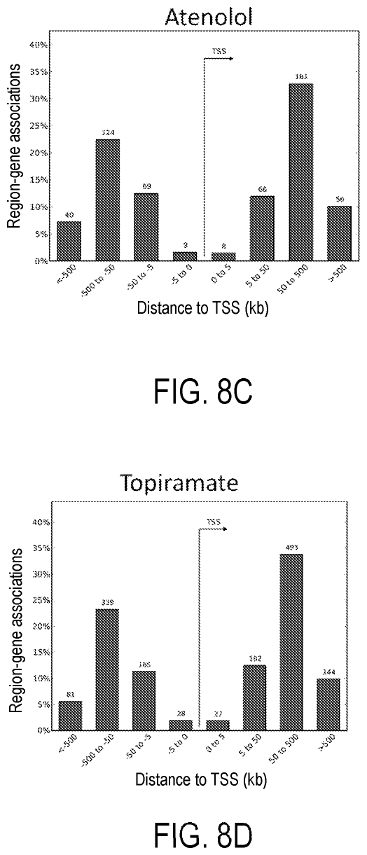

Association with treatment response reached genome-wide significance (p<5×10⁻⁸) in 374 variants for atenolol treatment and 1,031 variants for topiramate using PLINK with a general linear model. FIG. 8A illustrates a graph of results associated with Atenolol and FIG. 8B illustrates a graph of results associated with Topiramate.

Among the atenolol-associated variants, 289 (77%) were substitutions, 85 (23%) were insertions/deletions while in the topiramate-associated variants, 774 (75%) were substitutions and 257 (25%) were insertions/deletions. 22% (84) of atenolol and 26% (264) of topiramate treatment-associated SNPs were novel with no rs ID. No shared variants were identified between the atenolol and topiramate treatment groups. Additionally, none of the variants linked to either atenolol or topiramate response had been previously reported in migraine GWAS studies. Non-synonymous coding SNPs associated with treatment response were observed, including SNP (chr1:161365641:C/T:1) in CFAP126 for atenolol resulting in Arginine to Glutamine shift, SNP (chr7:100057239:T/G:1) in ZSCAN21 resulting in an Isoleucine to Serine shift, and SNP (chr12:122976756:G/A:1) in OGFOD2 resulting in an Aspartate to Asparagine shift for topiramate treatment (p<5×10⁻⁸).

The below table illustrates top genes ranked by the number of variants associated with atenolol treatment response.

| | No. of associated | Gene |
|---|---|---|
| Gene | variants | function |
| ZC3H12C | 33 | Enables endoribonuclease activity and mRNA |
| | | binding activity |
| C11orf87 | 18 | Integral component protein of membrane |
| RDX | 16 | Codes for a cytoskeletal protein that may be |
| | | important in linking actin to the plasma membrane |
| ELMOD1 | 10 | Involved in positive regulation of GTPase activity |
| SLN | 10 | Encodes a small proteolipid that regulates several |
| | | sarcoplasmic reticulum Ca(2+)-ATPases |
| ADGRL2 | 8 | Encodes a member of the latrophilin subfamily of G- |
| | | protein coupled receptors that participate in the |
| | | regulation of exocytosis |
| KBTBD8 | 6 | Involved in neural crest cell development; neural |
| | | crest formation; and protein monoubiquitination |
| PRICKLE2 | 6 | Encodes a homolog of Drosophila prickle and exact |
| | | function is not known, however, may be involved in |
| | | seizure prevention |
| PSMD6 | 6 | Encodes a member of the protease subunit S10 family |
| | | and is involved in the ATP-dependent degradation of |
| | | ubiquinated proteins |
| ADARB2 | 5 | Encodes a member of the double-stranded RNA |
| | | adenosine deaminase family of RNA-editing enzymes |
| | | and may play a regulatory role in RNA editing |
| FCGR3A | 5 | Encodes a receptor for the Fc portion of |
| | | immunoglobulin G, and it is involved in the removal |
| | | of antigen-antibody complexes from the circulation |
| HELZ | 5 | Member of the superfamily I class of RNA helicases |
| | | that alter the conformation of RNA by unwinding |
| | | double-stranded regions, thereby altering the biologic |
| | | activity of the RNA molecule and regulating access |
| | | to other proteins |

The below table illustrates top genes ranked by the number of variants associated with topiramate treatment response.

| | No. of associated | |
|---|---|---|
| Gene | variants | |
| OR4C46 | 15 | Encodes olfactory receptors interact with odorant |
| | | molecules in the nose, to initiate a neuronal response |
| | | that triggers the perception of a smell |
| CYP20A1 | 13 | CYP20A1 is a Cytochrome P450 protein involved in |
| | | drug metabolism |
| WDR12 | 13 | Encodes a member of the WD repeat protein family |
| | | that are involved in cell cycle progression, signal |
| | | transduction, apoptosis, and gene regulation |
| CDK6 | 11 | Encodes a member of the CMGC family of |
| | | serine/threonine protein kinases |
| TPTE | 10 | Encodes a PTEN-related tyrosine phosphatase which |
| | | may play a role in the signal transduction pathways of |
| | | the endocrine or spermatogenic function of the testis |
| ANO2 | 9 | Encodes calcium-activated chloride channels (CaCCs) |
| NTF3 | 9 | Encodes a member of the neurotrophin family, that |
| | | controls survival and differentiation of mammalian |
| | | neurons |
| TRAM1L1 | 9 | Involved in protein insertion into ER membrane |
| ZFP64 | 9 | Enables DNA binding activity and metal ion binding |
| | | activity |
| CWH43 | 8 | Involved in GPI anchor biosynthetic process |

FIGS. 8C and 8D illustrate the genomic distributions (with respect to Transcription Start Site) of variants associated with treatment response to Atenolol and Topiramate, respectively.

The below table describes pathways enriched (p<0.006) in SNPs identified as significantly associated with treatment response to Atenolol and Topiramate.

| Treatment | Pathway description | p-Value | Genes involved |
|---|---|---|---|
| Atenolol | Regulation of commissural axon | 0.000891 | ROBO2, SLIT3 |
| | pathfinding by SLIT and ROBO | | |
| | Citric acid cycle (TCA cycle) | 0.004416 | SDHC, SUCLG2 |
| | Defective ABCA12 in | 0.004548 | ABCA12 |
| | autosomal recessive congenital | | |
| | ichthyosis | | |
| Topiramate | Non-integrin membrane-ECM | 0.000632 | COL4A2, COL4A6, ITGAV, |
| | interactions | | PRKCA, TNC |
| | Synthesis of PIPs at the Golgi | 0.001146 | PIK3C2G, PIK3R4, TPTE |
| | membrane | | |
| | ECM proteogly cans | 0.001991 | COL4A2, COL4A6, IBSP, |
| | | | ITGAV, TNC |
| | Integrin cell surface interactions | 0.00325 | COL4A2, COL4A6, IBSP, |
| | | | ITGAV, TNC |
| | Syndecan interactions | 0.003804 | ITGAV, PRKCA, TNC |
| | ROBO receptors bind AKAP5 | 0.0047 | PRKCA, ROBO2 |
| | Laminin interactions | 0.005146 | COL4A2, COL4A6, ITGAV |
| | Regulation of commissural axon | 0.00583 | ROBO2, SLIT3 |
| | pathfinding by SLIT and ROBO | | |
| | Adaptive Immune System | 0.005936 | CTSS, ITGAV |

This example study employed WGS in the investigation of variants associated with therapeutic response in migraine prophylaxis. Prior GWAS studies in migraine were based on genotyping arrays, which is an approach biased in favor of targeting a limited number of common SNPs. The approach described above is less biased. It is contemplated that this was the first genome wide search for variants associated with therapeutic response in migraine prophylaxis to either Topiramate or Atenolol both of which are commonly prescribed in migraine treatment. Patients receiving monotherapy with each of these medications will continue to be sampled to form validation cohorts. WES data for subjects in whom quantitative therapeutic response data in migraine is retained may also be interrogated.

In the pathway most significantly associated with Atenolol response, the SLIT3 protein encoded by the altered gene is secreted, likely interacting with ROBO2 (roundabout) homolog receptors to effect cell migration and axon guidance. The second most implicated molecular cascade, citric acid cycle, is the main energy source for cells through its role in aerobic respiration. Of interest is the observation that the most significant pathway for Atenolol regulation of commissural axon pathfinding by SLIT and ROBO is also among the most significant pathways for Topiramate.

The most significant pathway associated with Topiramate response, non-integrin membrane-ECM interactions pathway is involved in extracellular matrix formation. The second most significant pathway drives the synthesis of phosphatidylinositol phosphates (PIPs) at the Golgi membrane. These biomolecules affect regulation of fundamental cellular processes, such as membrane trafficking and cytoskeleton remodeling.

It is contemplated that response to prophylactic treatment is an element of phenotype linked to the underlying relevant mechanism of action in migraine headache suppression.

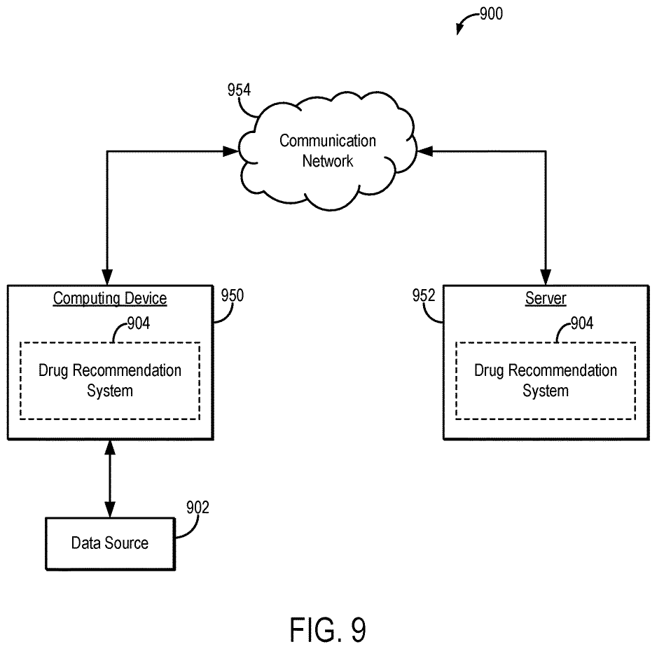

Referring now to FIG. 9, an example of a system 900 for generating or updating a candidate drug list of ranked and/or prioritized candidate drugs for treating a medical condition in a patient is shown. A computing device 950 can receive one or more types of data (e.g., patient health data) from data source 902. In some embodiments, computing device 950 can execute at least a portion of a drug recommendation system 904 to generate, from data received from the data source 902, a recommendation of an optimized candidate drug type and/or dosing to treat a particular medical condition in a particular patient. As described above, the recommendation may include generating and/or reprioritizing a list of candidate drugs for the patient based on their patient health data.

Additionally or alternatively, in some embodiments, the computing device 950 can communicate information about data received from the data source 902 to a server 952 over a communication network 954, which can execute at least a portion of the drug recommendation system 904. In such embodiments, the server 952 can return information to the computing device 950 (and/or any other suitable computing device) indicative of an output of the drug recommendation system 904.

In some embodiments, computing device 950 and/or server 952 can be any suitable computing device or combination of devices, such as a desktop computer, a laptop computer, a smartphone, a tablet computer, a wearable computer, a server computer, a virtual machine being executed by a physical computing device, and so on. The computing device 950 and/or server 952 can also reconstruct images from the data.

In some embodiments, data source 902 can be any suitable source of data (e.g., questionnaire response data, phenotypic data, genomics data, and other patient health data), such as one or more databases, another computing device (e.g., a server storing patient health data), and so on. In some embodiments, data source 902 can be local to computing device 950. For example, data source 902 can be incorporated with computing device 950 (e.g., computing device 950 can be configured as part of a device for measuring, recording, estimating, acquiring, or otherwise collecting or storing data). As another example, data source 902 can be connected to computing device 950 by a cable, a direct wireless link, and so on. Additionally or alternatively, in some embodiments, data source 902 can be located locally and/or remotely from computing device 950, and can communicate data to computing device 950 (and/or server 952) via a communication network (e.g., communication network 954).

In some embodiments, communication network 954 can be any suitable communication network or combination of communication networks. For example, communication network 954 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 954 can be a local area network, a wide area network, a public network (e.g., the Internet), a private or semi-private network (e.g., a corporate or university intranet), any other suitable type of network, or any suitable combination of networks. Communications links shown in FIG. 9 can each be any suitable communications link or combination of communications links, such as wired links, fiber optic links, Wi-Fi links, Bluetooth links, cellular links, and so on.

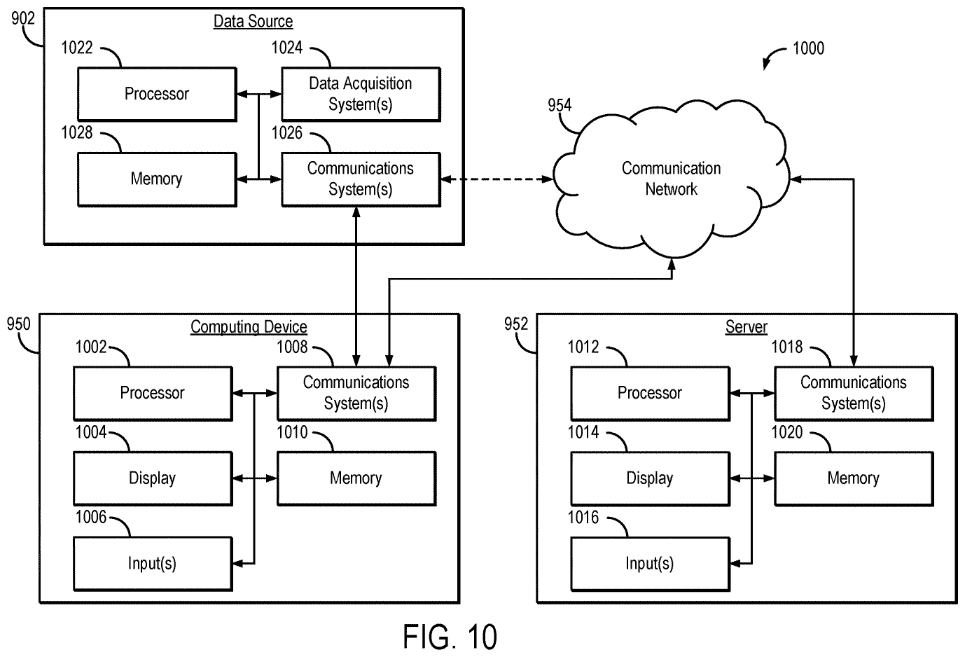

Referring now to FIG. 10, an example of hardware 1000 that can be used to implement data source 902, computing device 950, and server 952 in accordance with some embodiments of the systems and methods described in the present disclosure is shown.

As shown in FIG. 10, in some embodiments, computing device 950 can include a processor 1002, a display 1004, one or more inputs 1006, one or more communication systems 1008, and/or memory 1010. In some embodiments, processor 1002 can be any suitable hardware processor or combination of processors, such as a central processing unit (“CPU”), a graphics processing unit (“GPU”), and so on. In some embodiments, display 1004 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 1006 can include any suitable input devices and/or sensors that can be used to receive user input, such as a keyboard, a mouse, a touchscreen, a microphone, and so on.

In some embodiments, communications systems 1008 can include any suitable hardware, firmware, and/or software for communicating information over communication network 954 and/or any other suitable communication networks. For example, communications systems 1008 can include one or more transceivers, one or more communication chips and/or chip sets, and so on. In a more particular example, communications systems 1008 can include hardware, firmware, and/or software that can be used to establish a Wi-Fi connection, a Bluetooth connection, a cellular connection, an Ethernet connection, and so on.

In some embodiments, memory 1010 can include any suitable storage device or devices that can be used to store instructions, values, data, or the like, that can be used, for example, by processor 1002 to present content using display 1004, to communicate with server 952 via communications system(s) 1008, and so on. Memory 1010 can include any suitable volatile memory, non-volatile memory, storage, or any suitable combination thereof. For example, memory 1010 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 1010 can have encoded thereon, or otherwise stored therein, a computer program for controlling operation of computing device 950. In such embodiments, processor 1002 can execute at least a portion of the computer program to present content (e.g., images, user interfaces, graphics, tables), receive content from server 952, transmit information to server 952, and so on. For example, the processor 1002 and the memory 1010 can be configured to perform the methods described herein (e.g., the method of FIG. 3, the drug recommendation algorithms described herein, the drug recommendation optimizer algorithms described herein).

In some embodiments, server 952 can include a processor 1012, a display 1014, one or more inputs 1016, one or more communications systems 1018, and/or memory 1020. In some embodiments, processor 1012 can be any suitable hardware processor or combination of processors, such as a CPU, a GPU, and so on. In some embodiments, display 1014 can include any suitable display devices, such as an LCD screen, LED display, OLED display, electrophoretic display, a computer monitor, a touchscreen, a television, and so on. In some embodiments, inputs 1016 can include any suitable input devices and/or sensors that can be used to receive user input, such as a keyboard, a mouse, a touchscreen, a microphone, and so on.

In some embodiments, communications systems 1018 can include any suitable hardware, firmware, and/or software for communicating information over communication network 954 and/or any other suitable communication networks. For example, communications systems 1018 can include one or more transceivers, one or more communication chips and/or chip sets, and so on. In a more particular example, communications systems 1018 can include hardware, firmware, and/or software that can be used to establish a Wi-Fi connection, a Bluetooth connection, a cellular connection, an Ethernet connection, and so on.

In some embodiments, memory 1020 can include any suitable storage device or devices that can be used to store instructions, values, data, or the like, that can be used, for example, by processor 1012 to present content using display 1014, to communicate with one or more computing devices 950, and so on. Memory 1020 can include any suitable volatile memory, non-volatile memory, storage, or any suitable combination thereof. For example, memory 1020 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1020 can have encoded thereon a server program for controlling operation of server 952. In such embodiments, processor 1012 can execute at least a portion of the server program to transmit information and/or content (e.g., data, images, a user interface) to one or more computing devices 950, receive information and/or content from one or more computing devices 950, receive instructions from one or more devices (e.g., a personal computer, a laptop computer, a tablet computer, a smartphone), and so on.

In some embodiments, the server 952 is configured to perform the methods described in the present disclosure. For example, the processor 1012 and memory 1020 can be configured to perform the methods described herein (e.g., the method of FIG. 3, the drug recommendation algorithms described herein, the drug recommendation optimizer algorithms described herein).

In some embodiments, data source 902 can include a processor 1022, one or more data acquisition systems 1024, one or more communications systems 1026, and/or memory 1028. In some embodiments, processor 1022 can be any suitable hardware processor or combination of processors, such as a CPU, a GPU, and so on. In some embodiments, the one or more data acquisition systems 1024 are generally configured to acquire patient health data, and can include one or more client devices for presenting a user with a questionnaire and received response data, EMR systems, clinical laboratory systems or devices, and so on. Additionally, or alternatively, in some embodiments, the one or more data acquisition systems 1024 can include any suitable hardware, firmware, and/or software for coupling to and/or controlling operations of any suitable data acquisition systems. In some embodiments, one or more portions of the data acquisition system(s) 1024 can be removable and/or replaceable.

Note that, although not shown, data source 902 can include any suitable inputs and/or outputs. For example, data source 902 can include input devices and/or sensors that can be used to receive user input, such as a keyboard, a mouse, a touchscreen, a microphone, a trackpad, a trackball, and so on. As another example, data source 902 can include any suitable display devices, such as an LCD screen, an LED display, an OLED display, an electrophoretic display, a computer monitor, a touchscreen, a television, etc., one or more speakers, and so on.

In some embodiments, communications systems 1026 can include any suitable hardware, firmware, and/or software for communicating information to computing device 950 (and, in some embodiments, over communication network 954 and/or any other suitable communication networks). For example, communications systems 1026 can include one or more transceivers, one or more communication chips and/or chip sets, and so on. In a more particular example, communications systems 1026 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 1028 can include any suitable storage device or devices that can be used to store instructions, values, data, or the like, that can be used, for example, by processor 1022 to control the one or more data acquisition systems 1024, and/or receive data from the one or more data acquisition systems 1024; to generate images from data; present content (e.g., data, images, a user interface) using a display; communicate with one or more computing devices 950; and so on. Memory 1028 can include any suitable volatile memory, non-volatile memory, storage, or any suitable combination thereof. For example, memory 1028 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1028 can have encoded thereon, or otherwise stored therein, a program for controlling operation of data source 902. In such embodiments, processor 1022 can execute at least a portion of the program to generate images, transmit information and/or content (e.g., data, images, a user interface) to one or more computing devices 950, receive information and/or content from one or more computing devices 950, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the technology disclosed herein.

1. A method for generating an updated list of candidate drugs for treating a medical condition of a patient, the method comprising:
(a) accessing with a computer system, a list of candidate drugs for treating a medical condition;
(b) accessing with the computer system, patient health data acquired from a patient with the medical condition;
(c) accessing with the computer system, a machine learning algorithm that has been trained on training data to generate predictive scores for reprioritizing candidate drugs for treating the medical condition of the patient, wherein the predictive scores indicate at least one of a therapeutic efficacy of the candidate drugs, a safety of the candidate drugs, or a tolerability of the candidate drugs;
(d) inputting the patient health data to the machine learning algorithm using the computer system, generating output data as predictive scores for reprioritizing the list of candidate drugs; and
(e) inputting the list of candidate drugs and the predictive scores to a drug recommendation algorithm implemented by the computer system, generating an output as an updated list of candidate drugs that is optimized for the patient based on their patient health data.

2. The method of claim 1, wherein the machine learning algorithm is trained on training data using supervised learning.

3. The method of claim 1, wherein the patient health data comprise at least one of phenotypic data or genomic data.

4. The method of claim 3, wherein the patient health data comprise phenotypic data that include questionnaire response data indicating patient responses to a questionnaire.

5. The method of claim 4, wherein the medical condition is migraines or chronic headaches and the questionnaire response data comprise at least one of a headache frequency reported by the patient, a mean headache functional severity score reported by the patient, a mean headache pain intensity score reported by the patient, and a tolerability score reported by the patient, wherein the tolerability score indicates a tolerability of a presently prescribed drug for treating the medical condition.

6. The method of claim 1, wherein the predictive scores comprise at least one of positive predictive scores that increase a priority of an associated candidate drug in the list of candidate drugs and negative predictive scores that decrease a priority of an associated candidate drug in the list of candidate drugs.

7. The method of claim 1, wherein the machine learning algorithm is trained on a training data set comprising clusters of patient health data corresponding to a study group of patients having the medical condition, wherein the clusters of patient health data are correlated with different characteristics.

8. The method of claim 7, wherein the different characteristics comprise therapeutic efficacy of candidate drugs for treating the medical condition or tolerability of candidate drugs by patients in the study group of patients.

9. The method of claim 7, wherein the training data set is generated by inputting the patient health data for each patient in the study group of patients to an unsupervised learning algorithm, generating an output as the clusters of patient health data.

10. The method of claim 1, wherein the machine learning algorithm is a supervised learning-based machine learning algorithm.

11. The method of claim 10, wherein the supervised learning-based machine learning algorithm is a mixed models algorithm.

12. A method for generating a drug selection for treating a medical condition of a patient, the method comprising:
(a) accessing with a computer system, group medical data acquired from a plurality of patients associated with a group;
(b) accessing with the computer system, a first machine learning algorithm, wherein the first machine learning algorithm is an unsupervised learning algorithm;
(c) inputting the group medical data to the first machine learning algorithm using the computer system, generating cluster data as an output, wherein the cluster data comprise clusters of group medical data associated with a candidate drug characteristic;
(d) accessing with the computer system, new patient health data acquired from a new patient;
(e) accessing with the computer system, a second machine learning algorithm, wherein the second machine learning algorithm is a supervised learning algorithm;
(f) inputting the new patient health data and the cluster data to the second machine learning algorithm using the computer system, generating classified feature data as an output;
(g) accessing a drug recommendation model with the computer system, wherein the drug recommendation model is configured to determine a drug treatment recommendation based on patient health data;
(h) updating the drug recommendation model using the classified feature data; and
(i) inputting the new patient health data to the drug recommendation model, generating an optimized drug selection for the new patient.

13. A method for training a machine learning algorithm for reprioritizing a list of candidate drugs for treating a medical condition based on patient data acquired from a patient with the medical condition, the method comprising:
(a) generating, with a computer system, data clusters by inputting patient health data to a first machine learning algorithm using unsupervised learning, the patient health data being acquired from a group of subjects having the medical condition;
(b) training, with the computer system, a second machine learning algorithm on a second training data set using supervised learning, the second training data set comprising data clusters generated using the first machine learning algorithm, wherein the second machine learning algorithm is trained on the second training data set to generate classified feature data indicating a presence or absence of a characteristic in the data clusters that is indicative of at least one or therapeutic efficacy or tolerability of a candidate drug for treating the medical condition; and
(c) storing the second machine learning algorithm with the computer system.

14. The method of claim 13, wherein the group of subjects comprises subjects classified as one of responders or non-responders to at least one candidate drug included in the list of candidate drugs for treating the medical condition.

15. The method of claim 13, wherein the group of subjects comprises subjects classified as one of tolerators or non-tolerators of at least one candidate drug included in the list of candidate drugs for treating the medical condition.

16. The method of claim 13, wherein the first machine learning algorithm is a k-means clustering algorithm.

17. The method of claim 13, wherein the second machine learning algorithm is a mixed models algorithm.

18. A method for identifying a candidate drug for treating a medical condition of a subject, the method comprising:
(a) determining, with a computer system, functional pathway data as functional molecular cascades impacted by each of a plurality of candidate drugs for treating a medical condition of a subject;
(b) accessing, with the computer system, genomic data for the subject;
(c) determining, with the computer system, single nucleotide polymorphisms (SNPs) in the genomic data;
(d) determining, using the computer system, treatment pathway data comprising pathways affected by variations in the SNPs associated with at least one of treatment response or non-treatment response;
(e) determining an overlap of the functional pathway data and the treatment pathway data; and
(f) selecting a candidate drug for the subject based on the determined overlap.

19. The method of claim 18, wherein the functional pathway data are generated by an artificial intelligence-driven search for functional molecular cascaded impacted by each of the plurality of candidate drugs binding or non-receptor effects on direct and downstream effects.

20. The method of claim 18, wherein the treatment pathway data are generated based on determining from the genomic data, using the computer system, SNPs reaching a threshold associated with at least one of a positive outcome or a negative outcome with a treatment response for each of the plurality of candidate drugs.
